SEQUENCE LISTING (37 sequences)

SEQ ID NO: 1 - protein, 16 aa, synthetic construct
  REGION 1..16: note = Description of sequence: KSHV LANA peptide amino acid sequence
WKFAVIFWGNDPYGLK

SEQ ID NO: 2 - protein, 17 aa, synthetic construct
  REGION 1..17: note = Description of sequence: VGN73 peptide amino acid sequence
WKHAVIFWGNDPYGLKK

SEQ ID NO: 3 - protein, 16 aa, synthetic construct
  REGION 1..16: note = Description of sequence: VGN73 peptide amino acid sequence (variant1)
WKHAVIFWGNDPYGLK

SEQ ID NO: 4 - protein, 15 aa, synthetic construct
  REGION 1..15: note = Description of sequence: VGN73 peptide amino acid sequence (variant2)
WKHAVIFWGNDPYGL

SEQ ID NO: 5 - protein, 14 aa, synthetic construct
  REGION 1..14: note = Description of sequence: VGN73 peptide amino acid sequence (variant3)
WKHAVIFWGNDPYG

SEQ ID NO: 6 - protein, 13 aa, synthetic construct
  REGION 1..13: note = Description of sequence: VGN73 peptide amino acid sequence (variant4)
WKHAVIFWGNDPY

SEQ ID NO: 7 - protein, 12 aa, synthetic construct
  REGION 1..12: note = Description of sequence: VGN73 peptide amino acid sequence (variant5)
WKHAVIFWGNDP

SEQ ID NO: 8 - protein, 11 aa, synthetic construct
  REGION 1..11: note = Description of sequence: VGN73 peptide amino acid sequence (variant6)
WKHAVIFWGND

SEQ ID NO: 9 - protein, 12 aa, synthetic construct
  REGION 1..12: note = Description of sequence: TAT peptide amino acid sequence
GRKKRRQRRRPQ

SEQ ID NO: 10 - protein, 10 aa, synthetic construct
  REGION 1..10: note = Description of sequence: modified TAT amino acid sequence
  SITE 1: note = d-arginine
  MOD_RES 6: note = ORN
  MOD_RES 10: note = beta-alanine
RKKRRXRRRA

SEQ ID NO: 11 - protein, 26 aa, synthetic construct
  REGION 1..26: note = Description of sequence: TAT-KSHV LANA fusion peptide amino acid sequence
  SITE 1: note = d-arginine
  MOD_RES 6: note = ORN
  MOD_RES 10: note = beta-alanine
RKKRRXRRRAWKFAVIFWGNDPYGLK

SEQ ID NO: 12 - protein, 27 aa, synthetic construct
  REGION 1..27: note = Description of sequence: TAT-VGN73 fusion peptide amino acid sequence
  SITE 1: note = d-arginine
  MOD_RES 6: note = ORN
  MOD_RES 10: note = beta-alanine
RKKRRXRRRAWKHAVIFWGNDPYGLKK

SEQ ID NO: 13 - protein, 26 aa, synthetic construct
  REGION 1..26: note = Description of sequence: TAT-VGN73 variant1 fusion peptide amino acid sequence
  SITE 1: note = d-arginine
  MOD_RES 6: note = ORN
  MOD_RES 10: note = beta-alanine
RKKRRXRRRAWKHAVIFWGNDPYGLK

SEQ ID NO: 14 - protein, 25 aa, synthetic construct
  REGION 1..25: note = Description of sequence: TAT-VGN73 variant2 fusion peptide amino acid sequence
  SITE 1: note = d-arginine
  MOD_RES 6: note = ORN
  MOD_RES 10: note = beta-alanine
RKKRRXRRRAWKHAVIFWGNDPYGL

SEQ ID NO: 15 - protein, 24 aa, synthetic construct
  REGION 1..24: note = Description of sequence: TAT-VGN73 variant3 fusion peptide amino acid sequence
  SITE 1: note = d-arginine
  MOD_RES 6: note = ORN
  MOD_RES 10: note = beta-alanine
RKKRRXRRRAWKHAVIFWGNDPYG

SEQ ID NO: 16 - protein, 23 aa, synthetic construct
  REGION 1..23: note = Description of sequence: TAT-VGN73 variant4 fusion peptide amino acid sequence
  SITE 1: note = d-arginine
  MOD_RES 6: note = ORN
  MOD_RES 10: note = beta-alanine
RKKRRXRRRAWKHAVIFWGNDPY

SEQ ID NO: 17 - protein, 22 aa, synthetic construct
  REGION 1..22: note = Description of sequence: TAT-VGN73 variant5 fusion peptide amino acid sequence
  SITE 1: note = d-arginine
  MOD_RES 6: note = ORN
  MOD_RES 10: note = beta-alanine
RKKRRXRRRAWKHAVIFWGNDP

SEQ ID NO: 18 - protein, 21 aa, synthetic construct
  REGION 1..21: note = Description of sequence: TAT-VGN73 variant6 fusion peptide amino acid sequence
  SITE 1: note = d-arginine
  MOD_RES 6: note = ORN
  MOD_RES 10: note = beta-alanine
RKKRRXRRRAWKHAVIFWGND

SEQ ID NO: 19 - protein, 17 aa, synthetic construct
  REGION 1..17: note = Description of sequence: VGN73 mutant peptide amino acid sequence
AKHAVISAGNDPYGLKK

SEQ ID NO: 20 - protein, 27 aa, synthetic construct
  REGION 1..27: note = Description of sequence: TAT-VGN73 mutant fusion peptide amino acid sequence
  SITE 1: note = d-arginine
  MOD_RES 6: note = ORN
  MOD_RES 10: note = beta-alanine
RKKRRXRRRAAKHAVISAGNDPYGLKK

SEQ ID NO: 21 - protein, 23 aa, synthetic construct
  REGION 1..23: note = Description of sequence: LANA protein domain
EQELEEVEEQEEQELEEVEEQEQ

SEQ ID NO: 22 - protein, 21 aa, synthetic construct
  REGION 1..21: note = Description of sequence: LANA protein domain
EEVEEQEQQGVEQQEQETVEE

SEQ ID NO: 23 - protein, 23 aa, synthetic construct
  REGION 1..23: note = Description of sequence: LANA protein domain
ETVEEPIILHGSSSEDEMEVDYP

SEQ ID NO: 24 - protein, 23 aa, synthetic construct
  REGION 1..23: note = Description of sequence: LANA protein domain
EDEMEVDYPVVSTHEQIASSPPG

SEQ ID NO: 25 - protein, 24 aa, synthetic construct
  REGION 1..24: note = Description of sequence: LANA protein domain
IASSPPGDNTPDDDPQPGPSREYR

SEQ ID NO: 26 - protein, 23 aa, synthetic construct
  REGION 1..23: note = Description of sequence: LANA protein domain
GPSREYRYVLRTSPPHRPGVRMR

SEQ ID NO: 27 - protein, 21 aa, synthetic construct
  REGION 1..21: note = Description of sequence: LANA protein domain
RRVPVTHPKKPHPRYQQPPVP

SEQ ID NO: 28 - protein, 23 aa, synthetic construct
  REGION 1..23: note = Description of sequence: LANA protein domain
QPPVPYRQIDDPAKARPQHIFYR

SEQ ID NO: 29 - protein, 27 aa, synthetic construct
  REGION 1..27: note = Description of sequence: LANA protein domain
QHIIFYRRFLGKDGRRDPKCQWKFAVI

SEQ ID NO: 30 - protein, 27 aa, synthetic construct
  REGION 1..27: note = Description of sequence: LANA protein domain
KFAVIFWGNDPYGLKKLSQAFQFGGVK

SEQ ID NO: 31 - protein, 17 aa, Kaposi's sarcoma-associated herpesvirus
  REGION 1..17: note = Description of sequence: CHD4 binding region
WKFAVIFWGNDPYGLKK

SEQ ID NO: 32 - protein, 17 aa, macacine herpesvirus 5
  REGION 1..17: note = Description of sequence: CHD4 binding region
WRHGVIFCNSDPYSLYR

SEQ ID NO: 33 - protein, 17 aa, retroperitoneal fibromatosis-associated herpesvirus
  REGION 1..17: note = Description of sequence: CHD4 binding region
WRHAAIFWTPRPYPLKK

SEQ ID NO: 34 - protein, 17 aa, gamma herpesviruses 1
  REGION 1..17: note = Description of sequence: CHD4 binding region
WKHAALFWNPEPYPLKK

SEQ ID NO: 35 - protein, 18 aa, macacine herpesvirus 4
  REGION 1..18: note = Description of sequence: CHD4 binding region
WPFGVFYGPKTSCYNLRR

SEQ ID NO: 36 - protein, 20 aa, Epstein-Barr virus
  REGION 1..20: note = Description of sequence: CHD4 binding region
WVAGVFVYGGSKTSLYNLRR

SEQ ID NO: 37 - protein, 20 aa, saimiriine gammaherpesvirus 2
  REGION 1..20: note = Description of sequence: CHD4 binding region
WRHVVYFWGPTSALYRRLSK

(19) United States
(12) Patent Application Publication
(10) Pub. No.: US 2025/0282834 A1
(43) Pub. Date: Sep. 11, 2025

(54) PEPTIDE INHIBITORS FOR CHROMODOMAIN HELICASE DNA BINDING PROTEIN 4 (CHD4)

(71) Applicant: THE REGENTS OF THE UNIVERSITY OF CALIFORNIA, Oakland, CA (US)

(72) Inventors: Yoshihiro IZUMIYA, Oakland, CA (US); Kang-Hsin WANG, Oakland, CA (US); Hiroki MIURA, Oakland, CA (US)

(21) Appl. No.: 18/862,837

(22) PCT Filed: May 1, 2023
(86) PCT No.: PCT/US2023/020587

§ 371 Date: Nov. 4, 2024

Related U.S. Application Data
(60) Provisional application No. 63/337,792, filed on May 3, 2022.

(51) Int. Cl. C07K 14/005; A61K 31/403; A61K 38/00; A61P 35/00

(52) U.S. Cl. CPC C07K 14/005; A61K 31/403; A61P 35/00; A61K 38/00; C07K 2319/10; C07K 2319/50; C12N 2710/16422; C12N 2710/16433

(57) ABSTRACT

The present invention provides compositions and methods for treating a disease involving inappropriate or excessive proliferation of cells expressing CHD4.

### RELATED APPLICATIONS

This application claims priority to U.S. Provisional Patent Application No. 63/337,792, filed May 3, 2022, the contents of which are hereby incorporated in the entirety for all purposes.

### STATEMENT AS TO RIGHTS TO INVENTIONS MADE UNDER FEDERALLY SPONSORED RESEARCH AND DEVELOPMENT

This invention was made with Government support under grant numbers R01A1167663 awarded by the NIH NIAID. The Government has certain rights in the invention.

### BACKGROUND OF THE INVENTION

Viruses hijack host cell machinery for their replication, because they do not encode the necessary enzymes to replicate outside of infected host cells. They are thus molecular wizards, who control cellular functions, which includes cell apoptosis, cell cycle progression, as well as host immune responses. In viral replicating cells, host cell gene expression is frequently turned off, because the host transcriptional apparatus is diverted to transcribe viral genes. By studying which viral protein is responsible for controlling a specific host cell function, unique peptide drugs can be generated based on the viral protein sequence at the surface of the key viral-host protein interaction. Such peptide as well as its variants can be used as a dominant negative to inhibit the protein function.

On the other hand, the chromatin remodeling factor chromodomain helicase DNA-binding protein 4 (CHD4) is a core component of the nucleosome remodeling and deacetylase (NuRD) complex, and its oncogenic functions are beginning to be recognized in various cancer types by way of its negative effects on tumor suppressor genes. Due to its important role in DNA damage repair and transcription regulation, CHD4 has been identified as a key determinant in cancer progression, stem cell differentiation, and T cell and B cell development. Accumulating evidence reveals that CHD4 can function in NuRD-dependent and independent manners in response to DNA damage and gene regulation. For example, mutations of CHD4 have been shown to diminish its functions, which indicates that interpretation of its mutations may provide tangible benefit for patients. Further, the expression of CHD4 play a dual role in sensitizing cancer cells to chemotherapeutic agents, which provides new insights into the contribution of CHD4 to tumor biology and new therapeutic avenues.

Given the high prevalence of cancers, there exists a continuing need for new, safe, and effective treatments for targeting CHD4 to regulate the proliferation or activation of cells, especially in various lymphoproliferative disorders involving cancerous lymphoid cells such as B and T cells, in addition to other CHD4⁺ cancer cells. The present disclosure addresses this need and provides other advantages as well.

### BRIEF SUMMARY OF THE INVENTION

In the first aspect, the present invention provides a polypeptide comprising a CHD4-binding peptide and one or more heterologous amino acid sequences. The CHD4-binding peptide comprises the amino acid sequence set forth in any one of SEQ ID NOs:1-8, especially SEQ ID NOs:2-8, e.g., SEQ ID NO:2, is no more than about 20, 30, 50, or 100 amino acids in length, and inhibits the proliferation and/or suppresses the survival of a CHD4⁺ cell, especially a CHD4⁺ cancer cell such as leukemia or lymphoma cell. For instance, the CHD4-binding peptide may be no longer that about 16, 17, 18, 20, 25, 30, 35, 40, 45, 50, 55, 60, 65, 70, 80 or 90 amino acids. Further, the CHD4-binding peptide can be identified and screened for based on its ability to bind the CHD4 protein and/or to inhibit the proliferation or survival of CHD4⁺ cells.

In some embodiments, the CHD4-binding peptide comprises the amino acid sequence set forth in SEQ ID NO:2 and one or more additional peptides, preferably a heterologous peptide, such as a TAT sequence and/or an antibody or an antigen-binding fragment of an antibody. In some embodiments, the heterologous peptide is located at the N-terminus of the polypeptide. In some embodiments, the CHD4-binding peptide consists of the amino acid sequence set forth in SEQ ID NOs:1-8, for example, SEQ ID NO:s2-8, especially SEQ ID NO:2. In some cases, the polypeptide of this invention includes one or more D-amino acids, which may be located in the CHD4-binding peptide or the heterologous peptide(s), e.g., located at the N-terminus of the CH34-binding peptide and/or at the N-terminus of the polypeptide, and/or located at the C-terminus of the CHD4-binding peptide and/or at the C-terminus of the polypeptide. In some embodiments, the heterologous peptide is an antibody or an antigen-binding fragment thereof, for example, capable of specifically recognizing an antigen, such as a cell surface antigen naturally present on certain cell type of interest (e.g., CHD4⁺ cancer cells). In some embodiments, the antibody is a single chain antibody and/or is humanized. In some embodiments, the antigen recognized by the antibody is a cell surface antigen, such as one located on the surface of a CHD4⁺ tumor cell. In some embodiments, the CHD4-binding peptide and the heterologous peptide(s) are connected by a peptide linker, which in some cases includes one or more protease cleavage sites. In some embodiments, the polypeptide further includes a nuclear localization signal and % or a signal peptide at the N-terminus.

In some embodiments, the CHD4-binding peptide is a 16-amino-acid peptide, which has the activity of inhibiting proliferation of a cell or killing a cell that expresses CHD4 on the cell surface, especially a cancer cell. In some embodiments, the CHD4-binding peptide is a 17-amino-acid peptide. The peptide comprises the amino acid sequence set forth in the amino acid sequence SEQ ID NO:1, 2, 3, 4, 5, 6, 7, or 8. Further, the exemplary CHD4-binding peptide SEQ ID NO:1 may be modified according to at least one, possibly two or more, of the following possibilities: (a) at least one of the 16 amino acids is a D-amino acid (e.g., at position 1 or at the N-terminus of the peptide); (b) the amino acid sequence of the peptide differs from SEQ ID NO:1 at one or more positions 3, 5, 6, 9, 10, 11, and 13 by way of modification such as deletion, insertion, and/or substitution; or (c) the peptide is conjugated with a heterologous moiety attached to one or more amino acids within the peptide. For example, position 3 of SEQ ID NO:1 may be substituted with another amino acid. e.g., His (H). As a further example, a Lys (K) may be added to SEQ ID NO:1 at position 17.

In some embodiments, the CHD4-binding peptide has D-amino acid(s) at position 1 and/or position 2 of SEQ ID NO:1, 2, 3, 4, 5, 6, 7, or 8. In some embodiments, the heterologous moiety is a TAT peptide. For example, the TAT peptide and the CHD4-binding peptide are present within the same polypeptide chain. e.g., in the form of a fusion protein. An exemplary TAT peptide comprises or consists of the amino acid sequence of SEQ ID NO:8 or 9. In some embodiments, the N-terminal amino acid of the heterologous peptide is a D-amino acid. In some embodiments, the N-terminal amino acid of a fusion protein comprising a CHD4-binding peptide and one or more heterologous peptide is a D-amino acid.

In some embodiments, the peptide is conjugated with an antibody or an antibody fragment. For example, the antibody fragment is a single chain antibody such as a ScFv. In some embodiments, the peptide and the antibody (e.g., a single chain antibody) or antibody fragment are present within a single polypeptide chain as a fusion protein. In some embodiments, the antibody or antibody fragment is humanized. In some embodiments, the antibody or antibody fragment specifically recognizes or with a binding affinity for an antigen on the surface of a CHD4-expressing cell.

In some embodiments, the CHD4-binding peptide of SEQ ID NO:1 or a variant derived from SEQ ID NO:1 (such as any one of SEQ ID NOs:2-8. e.g., SEQ ID NO:2) is linked with a heterologous moiety by a chemical linker, for example, the CHD4-binding peptide is linked to a heterologous peptide by a peptide bond to form a fusion polypeptide. In some embodiments, the peptide of this invention further includes a nuclear localization signal. In some embodiments, the CHD4-binding peptide of SEQ ID NO:1 or derived from SEQ ID NO:1 (such as any one of SEQ ID NOs:2-8, e.g., SEQ ID NO:2) is linked with a polypeptide heterologous moiety by a cleavable peptide linker such as a linker containing one or more serine protease cleavage sites. In some embodiments, the fusion polypeptide of this invention has a cysteine residue at the C-terminus. In some embodiments, the fusion polypeptide of this invention has a signal peptide at the N-terminus.

In some embodiments, the heterologous moiety comprises a protein of a viral origin, such as a viral capsid protein (for example, an adenovirus or adeno-associated virus (AAV) or hepatitis E virus (HEV) capsid protein) or a portion thereof permitting formation of a virus-like particle (VLP). See. e.g., Büning and Srivastava. Mol Ther Methods Clin Dev. 12: 248-265 (2019); Le, et al., Sci Rep 9, 18631 (2019): U.S. Pat. No. 8,906,862: WO2019/178288; WO2019%236870. In other embodiments, the heterologous moiety comprises a virus (e.g., an adeno virus or AAV) or a VLP that contains a therapeutic DNA molecule, a therapeutic RNA molecule, a small molecule therapeutic agent, or any combination thereof.

In a second aspect, the present invention provides a nucleic acid comprising the polynucleotide sequence encoding the polypeptide of this invention in the form of a CHD4-binding peptide or in the form of a fusion protein comprising the CHD4-binding peptide, such as a peptide having the amino acid sequence of SEQ ID NO:1, 2, 3, 4, 5, 6, 7, or 8, comprising or consisting of the amino acid sequence of SEQ ID NO:1, 2, 3, 4, 5, 6, 7, or 8, optionally with one or more residues modified (e.g., substituted, deleted, or added), or a fusion protein between a CHD4-binding peptide and a second peptide derived from a heterologous origin.

In a third aspect, the present invention provides an expression cassette comprising a polynucleotide sequence encoding a CHD4-binding peptide or a fusion protein described above and herein, operably linked to a promoter, especially a heterologous promoter. Also provided is a vector comprising the polynucleotide sequence or the expression cassette. A host cell comprising the above-described nucleotide sequence encoding a CHD4-binding peptide or its fusion protein, a vector or an expression cassette comprising the nucleotide sequence, is also provided. In some cases, the host cell contains the CHD4-binding peptide or fusion protein.

In a related aspect, the present invention also provides a composition comprising a physiologically or pharmaceutically acceptable carrier or excipient along with the CHD4-binding peptide or peptide conjugate such as fusion protein of this invention, or a nucleic acid comprising a polynucleotide sequence encoding the CHD4-binding peptide or fusion protein, an expression cassette, or a vector comprising the coding sequence. In some embodiments, the composition comprising a physiologically or pharmaceutically acceptable carrier or excipient along with a host cell comprising the CHD4-binding peptide or the fusion protein of this invention, a nucleic acid comprising a polynucleotide sequence encoding the CHD4-binding peptide or fusion protein, or an expression cassette or a vector comprising the coding sequence for the CHD4-binding peptide or fusion protein. In some embodiments, the composition is a nanoparticle formulation of the CHD-4 binding peptide, fusion protein, or a nucleic acid encoding the peptide or fusion protein.

In a fourth aspect, the present invention provides a method for inhibiting proliferation of CDH4+ cells by contacting the cells with an effective amount of the polypeptide comprising a CHD4-binding peptide of this invention, optionally with one or more heterologous peptide, or a polynucleotide coding sequence for the polypeptide, or an effective amount of an expression cassette comprising the polynucleotide coding sequence for the polypeptide operably linked to a promoter directing the expression of the polypeptide, or a vector comprising such an expression cassette. In some embodiments, the cells are CHD4⁺ cancer cells, such as CHD4⁺ B or T cells.

In a related aspect, the present invention provides a method of treating a CDH4+ cancer in a subject in need thereof by administering to the subject an effective amount of (1) a polypeptide comprising a CHD4-binding peptide of this invention, optionally along with one or more heterologous peptide; (2) a polypeptide sequence encoding such a polypeptide; (3) an expression cassette comprising a polynucleotide coding sequence for the polypeptide operably linked to a promoter directing the expression of the polypeptide; (4) a vector (e.g., a viral vector) comprising such an expression cassette; and (5) host cells comprising such expression cassette or vector; or (6) a pharmaceutical composition comprising the polypeptide, the polynucleotide coding sequence, the expression cassette, the vector, or the host cells. In some embodiments, the cancer is leukemia or lymphoma, such as acute myeloid leukemia (AML), primary effusion lymphoma (PEL), or multiple myeloma. In some embodiments, the pharmaceutical composition used in the claimed method is a nanoparticle formulation of the CHD-4 binding peptide, fusion protein, or the nucleic acid encoding the peptide or fusion protein. In some embodiments, the treatment method includes having another anti-cancer chemotherapeutic agent (e.g., Sunitinib) currently administered to the subject.

### BRIEF DESCRIPTION OF THE DRAWINGS

FIG. 1. Identification of CHD4 binding domain. FIG. 1a. Schematic diagram of the KSHV LANA. LANA protein domain is depicted and non-structured regions (intrinsically unstructured region) were predicted by web interface (website: iupred2a.elte.hu/) and plotted underneath of the diagram. Previously-identified CHD4 binding region were enlarged. FIG. 1b. Tiling N-biotinylated peptide sequence. Amino acid position is shown. FIG. 1c. Peptide pull-down assays. One hundred nano molar of purified CHD4 protein was incubated with 1 μg of biotinylated peptides. Peptide bound Flag-CHD4 was visualized by immunoblotting with anti-Flag antibody. FIG. 1d. Position of CHD4 binding domain. Previously revealed crystal structure of LANA C-terminal domain was used as a reference to visualize the position of CHD4 binding region. Each monomer of LANA C-terminal domain were visualized with gray (left-side of dimer) and pink (right-side), respectively. The protein backbone and side-chains of CHD4 binding region on the LANA monomer (left-side) was highlighted with Green. FIG. 1e. Protein sequence alignments. Conserved amino acids was revealed by aligning with other gamma herpesvirus homologs. Conserved amino acids was depicted bold case and similar amino acids are marked in italic. Designed peptide based on LANA protein sequence was shown in bottom. Underlining amino acid residue was substituted with histidine for better cell membrane penetration.

FIG. 2 VGN73 binding with CHD4 and LANA. FIG. 2a. Analysis of VGN73 binding region in CHD4 by ELISA. Increasing concentrations of Flag conjugated CHD4 were incubated in triplicate in an ELISA plate coated with 1 μM of VGN73. Peptide binding measured as OD values at 450 nm are shown. FIG. 2b. Sequences of three peptides used for biolayer interferometry (BLI). From top to bottom: SEQ ID NO:12; SEQ ID NO:20; and SEQ ID NO:10. The underline indicates three amino acids changes at conserved amino acids in the mutant peptide. FIG. 2c. Profiling binding kinetics of VGN73 and mutant to CHD4, and reference control TAT to CHD4 by BLI. Binding kinetics was evaluated using a 1:1 binding model by ForteBio Data Analysis 8.1 software. FIG. 2d. Analysis of subcellular peptide localization by Immunofluorescence assay. Cy5-conjugated VGN73 and mutant peptide were used to track subcellular localization (15 min or 60 min) in BC3 cells. Nuclei were visualized by Hoechst 33342 staining. The scale is indicated in the panel. FIG. 2e. Evaluating that VGN73 binds CHD4 and LANA in tissue culture by peptide-pull down assay with streptavidin beads. BC3 cell were incubated with biotinylated VGN73, biotinylated mutant peptide or biotinylated TAT peptide (10 μM of each) for 24 hours. TAT alone and 0.5% of the input reaction before peptide-pull down was used as control. The authenticity of respective protein band was confirmed by immunoblotting with specific antibodies.

FIG. 3 Effect of VGN73 on CHD4 and LANA protein in tissue culture. FIG. 3. Amount of CHD4 and LANA protein expression was evaluated by IFA. BC3 cells were treated with VGN73 (18 μM) or mutant peptide (18 μM) for 24 hours. Then the cells were stained with the indicated antibodies. The scale is indicated in the panel. Cell volume. CHD4 and LANA signal intensity were compared. **; p<0.01, ***; p<0.001 versus the VGN73 group. FIG. 3b. Amount of CHD4 and LANA protein expression in total cell lysates was evaluated by western blotting. BC3 and BCBL-1 cells were treated with VGN73 at a concentration of IC50 or starved condition for 24 hours. The respective protein band was confirmed by immunoblotting with anti-CHD4 and anti-LANA antibody. FIG. 3c. Evaluating of caspase and proteasome in CHD4 cleavage by VGN73. BC3 cells were pretreated with pan-caspase inhibitor. Z-VAD-FMK (50 μM) or proteasome inhibitor, bortezomib (25 nM) for 1 hour, followed by treated with VGN73 (18 μM) for 8 hours. The respective protein band was confirmed by immunoblotting with anti-CHD4 antibody.

FIG. 4 VGN73 inhibits cell growth on various cancer cell types and induces apoptosis and autophagy in BC3 cells. FIG. 4a. MT assays were performed with the indicated cell lines treated with various VGN73 and mutant peptide concentrations for 24 hours. The OD of mock-treated samples were set as 100%, and OD from detergent-treated cells were set as 0%. Mean percentage viability±SD was calculated for each treatment (n=3 samples/treatment). FIG. 4b. VGN73 induces apoptosis. BC3 cells were incubated with of VGN73 (18 μM) for 0, 1, 4, and 24 hours, followed by measurements of percentage apoptosis induction (Annexin V staining) by flow cytometry. Median percentage apoptosis was calculated for each treatment time (n=3 samples/treatment time) (a). Western blot analysis was performed to detect cleaved caspase 3. BC3 cells were with VGN73 or mutant peptide (18 μM) for 24 hours. Cleaved caspase 3 were calculated by normalizing to untreated sample, and untreated sample set as 100% (b). FIG. 4c. VGN73 induces autophagy. BC3 cells were incubated with 18 μM of VGN73. 50 nM of rapamycin and starved condition for 24 hours were used positive controls. Flow cytometry analyses (Autophagy red staining) were performed. Median percentage apoptosis was calculated for each treatment time (n=3 samples/treatment) (a). Western blot analysis was performed to detect LC3II and LC3I. BC3 cells were pretreated with lysosome inhibitor, chloroquine (7.5 μM) for 1 hr. LC3I/LC3I ratios were calculated by normalizing to untreated sample, and untreated sample set as 100% (b). FIG. 4d. Pan caspase inhibitor inhibits cell death by VGN73. BC3 and BCBL1 cells were pretreated with Z-VAD-FMK (40 μM) for 1 hr and then incubated VGN73 (18 or 13 μM, respectively) for 8 hours. Cell viability were determined by MTT assay (n=5 samples/treatment). *; p<0.05. **; p<0.01 versus the control group, #; p<0.05 versus the Z-VAD-FMK group.

FIG. 5 VGN73 enhances U937 cells differentiation. FIG. 5a. The scheme that total RINA-sequencing of U937 differentiation by pretreating with VGN73. U937 cells were pretreated with 0, 4, or 8 μM VGN73 for 24 hours, then stimulated with PMA at a final concentration of 10 ng/mL for another 24 hours. Red; treated with PMA alone, Green: treated with VGN73 alone. Blue and Purple; pretreated with VGN73 (4 or 8 μM, respectively) and stimulated with PMA. FIG. 5b. Heatmap showing the relative levels of gene expression between each treatment by total RNA-sequencing. Difference of gene set between PMA alone and VGN73 alone was marked with asterisks. FIG. 5c. Evaluation of representative gene expression which are associated with macrophage differentiation and inflammatory markers by total RNA-sequencing. Macrophage differentiation markers and inflammatory associated genes were especially increased in VGN73-pretreated and PMA-stimulated U937 cells. FIG. 5d. Change in macrophage or dendritic cell surface marker by Flow cytometry. Red; non-treated cell, Blue; treated with 4 μM of VGN73 for 5 days, Orange; without VGN pretreated cells stimulating with 10 ng/mL PMA for 3 days. Green; pretreating cells with 4 μM of VGN73 for 2 days, then stimulating with 10 ng/mL of PMA for 3 days.

FIG. 6 VGN73 Inhibits BCBL-1 cell growth in xenograft mice. BCBL-1 cells (5×10⁶ cells) were injected into intraperitoneal of 23-30 weeks old female NRG mice. After 2 days, mice were randomly assigned to 3 treatment groups: PBS, mutant peptide (10 mg/kg), and VGN73 (10 mg/kg) (n=6 mice per group). Treatment drugs were administrated i.p. every other day. FIG. 6a. In vivo bioluminescence imaging. The tumors at day 14 and 19 were measured by in vivo bioluminescence imaging (n=6 mice per group), and these intensities were compared among each group. FIG. 6b. Tumor growth was monitored by measurement of body weight. Mice were weighed every other day for 18 days and body weight increments were compared among each group at day 14 and 18 (n=6 mice per group). FIG. 6c. PEL cell counts in ascites fluid were measured at day 19 by flow cytometry (n=6 mice per group). FIG. 6d. Expression of CHD4 and LANA protein in ascites cells after treating. Cells were stained with the indicated antibodies. The scale is indicated in the panel. FIG. 6e. Gene expression of cell surface marker on PEL cells recovered from ascites after treating. CD38 and CD19 gene expression was quantified by RT-qPCR. 18S ribosomal RNA was used as an internal standard to normalize viral gene expression, a-c and e *; p<0.05, **; p<0.01.

FIG. 7a FLAG-tagged CHD4 deletion proteins. These proteins were prepared from recombinant baculovirus infected Sf9 cells. Coommasie blue staining is shown. The CHD4 deletion proteins were used to determine the peptide binding sites described in FIG. 2a. FIG. 7b FLAG-tagged CHD4 and LANA protein. These proteins were prepared from recombinant baculovirus infected Sf9 cells. Coommasie blue staining is shown. The full-length CHD4 and LANA were used in studies presented in FIGS. 2b and 2c. FIG. 7c Evaluation of VGN73 in LANA interaction with CHD4. (a) A schematic diagram of peptide-pull down assay. (b) Peptide-pull-down assay. CHD4 (200 nM) and LANA (200 nM) were incubated with VGN73 (1 or 10 μM) or mutant peptide (1 or 10 μM) in 200 μL binding buffer. 5% of the input reaction before peptide-pull down was used as control. The precipitated proteins were confirmed by immunoblotting with anti-CHD4 and anti-LANA antibody. (c) Putative model of VGN73 actions in vitro.

FIG. 8a Selective protein cleavages by VGN73 incubation. Amount of BRM, SMARCE1 and IRF4 protein expression in total cell lysates was examined by western blotting. BC3 and BCBL-1 cells were treated with VGN73 at a concentration of IC50 for 24 hours. The indicted protein expression was confirmed by immunoblotting with specific antibody. FIG. 8b. Gene expression after VGN73 treatment in BC3 cells. Human CHD4, LANA and K-Rta gene expression was quantified by RT-qPCR. 18S ribosomal RNA was used as an internal standard to normalize viral gene expression. The VGN73 peptide induces KSHV reactivation in KSHV latently infected PEL cells. *: p<0.05.

FIG. 9. Direct target identification with SLAM-seq. PBS, VGN73, or mutant peptide at each concentration of IC50 were added to BC3, BCBL-1 or Raji cells 30 mins prior to incubation with 4sU, and RNA was labeled for 1.5 hrs in the presence of each peptide. Each treatment was performed in duplicate. FIG. 9a. Principal component analysis of SLAM-seq from each cell lines treated with PBS, Mutant or VGN73. FIG. 9b. Venn diagram of up-regulated genes. Venn diagram shows commonly upregulated gene sets by VGN73 incubation comparing with PBS among BC3. BCBL-1, and Raji cells. FIG. 9c. Cleavage under targets & release using nuclease with the CHD4 antibodies. Peak scores of promoter region were compared between upregulated gene and non-upregulated gene in SLAM-seq data and depicted with violin plots. d. CUT&RUN with qPCR. To confirm the occupancies of CHD4 on promoter regions of upregulated gene, CUT&RUN-qPCR was performed with CHD4 antibody in BCBL-1 cells. Each promoter region was compared with negative control genomic locus. FIG. 9e. VGN73 decreases CHD4 occupancies on promoters. CUT&RUN with qPCR was performed with the selected promoter regions. Ratio between VGN73 treated and non-treated samples was presented. VGN73 incubation decreased the occupancies of CHD4 from the promoter. NC; negative control genomic locus, *; p<0.05 versus the ctrl.

FIG. 10. Morphological change in macrophage differentiation with or without VGN73 pretreating was evaluated by IFA. U937 cells were pretreated with VGN73 (4 μM) for 24 hours, followed by stimulated with PMA (10 ng/mL) in starved condition for 72 hrs. Then the cells were stained with the indicated antibodies.

FIG. 11a. Kaplan-Meier survival curve. Maximum tolerate dose with NRG mice was determined prior xenograft studies. The 11-13 weeks old mice (female; n=4, male; n=4) were injected with increasing amount of VGN73 intraperitoneally every other day. FIG. 11b. Toxicology studies. BALB/c mice (8 weeks old, male; n=6, female; n=6/group) were injected with PBS. 10 mg/kg of VGN73 or mutant peptide intraperitoneally every other day for 18 days. (a) Body weight change Body weight changes were measured every other day. (b) Serum biochemistry. At the end of the experiment, fresh serum collected were subjected for serum biochemical analyses.

### DEFINITIONS

The terms “a,” “an,” or “the” as used herein not only include aspects with one member, but also include aspects with more than one member. For instance, the singular forms “a,” “an,” and “the” include plural referents unless the context clearly dictates otherwise. Thus, for example, reference to “a cell” includes a plurality of such cells and reference to “the agent” includes reference to one or more agents known to those skilled in the art, and so forth.

The terms “about” and “approximately” as used herein denotes a range of ±10% of a pre-determined value. For example. “about 10” indicates a range of 10±1. i.e., 9-11.

The term “nucleic acid sequence encoding a peptide” refers to a segment of DNA, which in some embodiments may be a gene or a portion thereof, that is involved in producing a peptide chain (e.g., an antigen or fusion protein). A gene will generally include regions preceding and following the coding region (leader and trailer) involved in the transcription/translation of the gene product and the regulation of the transcription/translation. A gene can also include intervening sequences (introns) between individual coding segments (exons). Leaders, trailers, and introns can include regulatory elements that are necessary during the transcription and the translation of a gene (e.g., promoters, terminators, translational regulatory sequences such as ribosome binding sites and internal ribosome entry sites, enhancers, silencers, insulators, boundary elements, replication origins, matrix attachment sites and locus control regions, etc.). A “gene product” can refer to either the mRNA or protein expressed from a particular gene.

The terms “expression” and “expressed” refer to the production of a transcriptional and/or translational product, e.g., of a nucleic acid sequence encoding a protein (such as a CHD4-inhibiting peptide or fusion protein). In some embodiments, the term refers to the production of a transcriptional and/or translational product encoded by a gene (e.g., a gene encoding a CHD4-inhibiting peptide or fusion protein) or a portion thereof. The level of expression of a DNA molecule in a cell may be assessed on the basis of either the amount of corresponding mRNA that is present within the cell or the amount of protein encoded by that DNA produced by the cell.

The term “recombinant” when used in reference, e.g., to a polynucleotide, protein, vector, or cell, indicates that the polynucleotide, protein, vector, or cell has been modified by the introduction of a heterologous nucleic acid or protein or the alteration of a native nucleic acid or protein, or that the cell is derived from a cell so modified. For example, recombinant polynucleotides contain nucleic acid sequences that are not found within the native (naturally occurring) form of the polynucleotide.

As used herein, the terms “polynucleotide” and “nucleic acid” refer to deoxyribonucleic acids (DNA) or ribonucleic acids (RNA) and polymers thereof. The term includes, but is not limited to, single-, double-, or multi-stranded DNA or RNA, genomic DNA, cDNA, and DNA-RNA hybrids, as well as other polymers comprising purine and/or pyrimidine bases or other natural, chemically modified, biochemically modified, non-natural, synthetic, or derivatized nucleotide bases. Unless specifically limited, the term encompasses nucleic acids containing known analogs of natural nucleotides that have similar binding properties as the reference nucleic acid. Unless otherwise indicated, a particular nucleic acid sequence also implicitly encompasses conservatively modified variants thereof (e.g., degenerate codon substitutions), homologs, and complementary sequences as well as the sequence explicitly indicated. Specifically, degenerate codon substitutions may be achieved by generating sequences in which the third position of one or more selected (or all) codons is substituted with mixed-base and/or deoxyinosine residues (Batzer et al., Nucleic Acid Res. 19:5081 (1991); Ohtsuka et al., J. Biol. Chem. 260:2605-2608 (1985); and Rossolini et al., Mol. Cell. Probes 8:91-98 (1994)).

The terms “vector” and “expression vector” refer to a nucleic acid construct, generated recombinantly or synthetically, with a series of specified nucleic acid elements that permit transcription of a particular nucleic acid sequence (e.g., encoding an antigen and/or fusion protein of the invention) in a host cell or engineered cell. In some embodiments, a vector includes a polynucleotide to be transcribed, operably linked to a promoter. Other elements that may be present in a vector include those that enhance transcription (e.g., enhancers), those that terminate transcription (e.g., terminators), those that confer certain binding affinity or antigenicity to a protein (e.g., recombinant protein) produced from the vector, and those that enable replication of the vector and its packaging (e.g., into a viral particle). In some embodiments, the vector is a viral vector (i.e., a viral genome or a portion thereof). A vector may contain nucleic acid sequences or mutations, for example, that increase tropism and/or modulate immune function. An “expression cassette” comprises a coding sequence, operably linked to a promoter, and optionally a polyadenylation sequence.

The terms “polypeptide,” “peptide,” and “protein” are used interchangeably herein to refer to a polymer of amino acid residues. All three terms apply to amino acid polymers in which one or more amino acid residues are an artificial chemical mimetic of a corresponding naturally occurring amino acid, as well as to naturally occurring amino acid polymers and non-naturally occurring amino acid polymers. As used herein, the terms encompass amino acid chains of any length, including full-length proteins, wherein the amino acid residues are linked by covalent peptide bonds.

The terms “subject,” “individual,” and “patient” are used interchangeably herein to refer to a vertebrate, preferably a mammal, more preferably a human. Mammals include, but are not limited to, rodents (mice, rats, etc.), felines, bovines, simians, primates (including humans), farm animals, sport animals, and pets. Tissues, cells and their progeny of a biological entity obtained in vivo or cultured in vitro are also encompassed.

As used herein, the term “administering” includes oral administration, topical contact, injection of various modes and other means of administration as a suppository, intravenous, intraperitoneal, intramuscular, intralesional, intratumoral, intrathecal, intranasal, intraosseous, or subcutaneous administration to a subject. Administration is by any route, including parenteral and transmucosal (e.g., buccal, sublingual, palatal, gingival, nasal, vaginal, rectal, or transdermal). Parenteral administration includes, e.g., intravenous, intramuscular, intra-arterial, intradermal, subcutaneous, intraperitoneal, intraventricular, intraosseous, and intracranial. Other modes of delivery include, but are not limited to, the use of liposomal formulations, intravenous infusion, transdermal patches, etc.

The term “treating” refers to an approach for obtaining beneficial or desired results including, but not limited to, a therapeutic benefit and/or a prophylactic benefit. “Therapeutic benefit” means any therapeutically relevant improvement in or effect on one or more diseases, conditions, or symptoms under treatment. Therapeutic benefit can also mean to effect a cure of one or more diseases, conditions, or symptoms under treatment. Furthermore, therapeutic benefit can also mean to increase survival. For prophylactic benefit, the compositions may be administered to a subject at risk of developing a particular disease, condition, or symptom, or to a subject reporting one or more of the physiological symptoms of a disease, even though the disease, condition, or symptom may not yet be present.

The term “therapeutically effective amount” or “sufficient amount” refers to the amount of a system, recombinant polynucleotide, or composition described herein that is sufficient to effect beneficial or desired results. The therapeutically effective amount may vary depending upon one or more of: the subject and disease condition being treated, the weight and age of the subject, the severity of the disease condition, the immune status of the subject, the manner of administration and the like, which can readily be determined by one of ordinary skill in the art. The specific amount may vary depending on one or more of: the particular agent chosen, the target cell type, the location of the target cell in the subject, the dosing regimen to be followed, whether it is administered in combination with other compounds, timing of administration, and the physical delivery system in which it is carried.

For the purposes herein an effective amount is determined by such considerations as may be known in the art. The amount must be effective to achieve the desired therapeutic effect in a subject suffering from a disease such as an infectious disease or cancer. The desired therapeutic effect may include, for example, amelioration of undesired symptoms associated with the disease, prevention of the manifestation of such symptoms before they occur, slowing down the progression of symptoms associated with the disease, slowing down or limiting any irreversible damage caused by the disease, lessening the severity of or curing the disease, or improving the survival rate or providing more rapid recovery from the disease. Further, in the context of prophylactic treatment the amount may also be effective to prevent the development of the disease.

The term “pharmaceutically acceptable carrier” refers to a substance that aids the administration of an active agent to a cell, an organism, or a subject. “Pharmaceutically acceptable carrier” also refers to a carrier or excipient that can be included in the compositions of the invention and that causes no significant adverse toxicological effect on the patient. Non-limiting examples of pharmaceutically acceptable carriers include water, sodium chloride (NaCl), normal saline solutions, lactated Ringer's, normal sucrose, normal glucose, binders, fillers, disintegrants, lubricants, coatings, sweeteners, flavors and colors, liposomes, dispersion media, microcapsules, cationic lipid carriers, isotonic and absorption delaying agents, and the like. The carrier may also comprise or consist of substances for providing the formulation with stability, sterility and isotonicity (e.g. antimicrobial preservatives, antioxidants, chelating agents and buffers), for preventing the action of microorganisms (e.g. antimicrobial and antifungal agents, such as parabens, chlorobutanol, phenol, sorbic acid and the like) or for providing the formulation with an edible flavor, etc. In some instances, the carrier is an agent that facilitates the delivery of a polypeptide, fusion protein, or polynucleotide to a target cell or tissue. One of skill in the art will recognize that other pharmaceutical carriers are useful in the present invention.

The phrase “specifically binds” refers to a molecule (e.g., a peptide or a protein especially an antibody or antibody fragment against a cancer cell antigen) that binds to a target with greater affinity, avidity, more readily, and/or with greater duration to that target in a sample than it binds to a non-target compound. In some embodiments, a molecule that specifically binds a target binds to the target with at least 2-fold greater affinity than non-target compounds, e.g., at least 3-fold, 4-fold, 5-fold, 6-fold, 7-fold, 8-fold, 9-fold, 10-fold, 20-fold, 25-fold, 50-fold or greater affinity.

As used in herein, the terms “identical” or percent “identity,” in the context of describing two or more polynucleotide or amino acid sequences, refer to two or more sequences or specified subsequences that are the same. Two sequences that are “substantially identical” have at least 60% identity, preferably 65%, 70%, 75%, 80%, 85%, 90%, 91%, 92%, 93, 94%, 95%, 96%, 97%, 98%, 99%, or 100% identity, when compared and aligned for maximum correspondence over a comparison window, or designated region as measured using a sequence comparison algorithm or by manual alignment and visual inspection where a specific region is not designated. With regard to polynucleotide sequences, this definition also refers to the complement of a test sequence. With regard to amino acid sequences, in some cases, the identity exists over a region that is at least about 50 amino acids or nucleotides in length, or more preferably over a region that is 75-100 amino acids or nucleotides in length.

For sequence comparison, typically one sequence acts as a reference sequence, to which test sequences are compared. When using a sequence comparison algorithm, test and reference sequences are entered into a computer, subsequence coordinates are designated, if necessary, and sequence algorithm program parameters are designated. Default program parameters can be used, or alternative parameters can be designated. The sequence comparison algorithm then calculates the percent sequence identities for the test sequences relative to the reference sequence, based on the program parameters. For sequence comparison of nucleic acids and proteins, the BLAST 2.0 algorithm and the default parameters are used.

The term “heterologous” as used in the context of describing the relative location of two elements, refers to the two elements such as polynucleotide sequences (e.g., a promoter or a protein/polypeptide-encoding sequence) or polypeptide sequences (e.g., any one of SEQ ID NOs:1-8, especially SEQ ID NOs:2-8, and another peptide sequence serving as a fusion partner with, e.g., SEQ ID NO:2, in a conjugate in the form of a fusion polypeptide) that are not naturally found in the same relative positions. Thus, a “heterologous promoter” of a gene refers to a promoter that is not naturally operably linked to that gene. Similarly, a “heterologous polypeptide” or “heterologous polynucleotide” to SEQ ID NO:1 or its encoding sequence is one derived from an origin different from the protein of which SEQ ID NO:1 is a naturally-occurring fragment. The fusion of SEQ ID NO:1 (or its coding sequence) with a heterologous polypeptide (or polynucleotide sequence) does not result in a longer polypeptide or polynucleotide sequence that can be found in nature as an intact protein (or its coding sequence) or a segment thereof.

When used in the context of describing a conjugate comprising the CHD4-binding peptide of SEQ ID NO:1 or a derivative thereof (such as any one of SEQ ID NOs:2-8, especially SEQ ID NO:2), the term “heterologous moiety” refers to a conjugation partner of the CHD4-binding peptide as one originated from a source other than ORF50 protein of the Kaposi's sarcoma-associated herpesvirus (KSHV). In some embodiments, the a CHD4-binding peptide is a peptide slightly longer than any one of SEQ ID NOs:1-8, for example, it may consist of a core peptide having the amino acid sequence set forth in any one of SEQ ID NOs:1-8 (e.g., SEQ ID NO:2) plus an additional 1, 2, 3, 4, 5, 6, 7, 8, 9, 10 or up to 11 or 12 amin acids at either or both of the N-terminus and C-terminus of the core peptide. In embodiments where the conjugate of the CHD4-binding peptide and the heterologous moiety is a fusion protein, i.e., the heterologous moiety being another polypeptide and fused to the CHD4-binding peptide via a peptide bond, the fusion of two peptide partners should not result in the full length KSHV ORF50 protein and preferably not result in a segment of the KSHV ORF50 protein significantly longer than SEQ ID NO:1. e.g., a segment of more than 16, 17, 18, 19, 20, 25, or 30 amino acids in length. In some embodiments, such a fusion protein comprises any one of SEQ ID NOs:2-8 (e.g., SEQ ID NO:2) and is no longer than 16, 17, 18, 19, 20, 25, 30, 35, 37, 40, 45, 50, 60, 70, 80, 90, or 100 amino acids in length. In some embodiments, the heterologous moiety may be one with therapeutic efficacy. e.g., the capability to cause death of target cells either by direct killing or by triggering programmed cell death (apoptosis). Such a therapeutic moiety may be a polypeptide in nature (e.g., an antibody, such as an anti-CD3 antibody, especially a single chain antibody ScFv) or a non-polypeptide (e.g., a cytotoxic agent in the form of a carbohydrate or oligonucleotide). In other embodiments, the heterologous moiety may be non-therapeutic in nature but serves as an affinity moiety, a targeting moiety, a detectable/signal moiety, or a solid support or provides other utilities so as to facilitate the detection, isolation, purification, tissue/cell-targeted delivery, and/or immobilization of the conjugate comprising the peptide of SEQ ID NO:1 or a derivative thereof (e.g., SEQ ID NOs:2-8, especially SEQ ID NO:2).

The term “cancer” refers to any of various malignant neoplasms characterized by the proliferation of anaplastic cells that tend to invade surrounding tissue and metastasize to new body sites. Non-limiting examples of different types of cancer suitable for treatment using the compositions and methods of the present invention include colorectal cancer, colon cancer, anal cancer, liver cancer, ovarian cancer, breast cancer, lung cancer, bladder cancer, thyroid cancer, pleural cancer, pancreatic cancer, cervical cancer, prostate cancer, testicular cancer, bile duct cancer, gastrointestinal carcinoid tumors, esophageal cancer, gall bladder cancer, rectal cancer, appendix cancer, small intestine cancer, stomach (gastric) cancer, renal cancer (e.g., renal cell carcinoma), cancer of the central nervous system, skin cancer, oral squamous cell carcinoma, choriocarcinomas, head and neck cancers, bone cancer, osteogenic sarcomas, fibrosarcoma, neuroblastoma, glioma, melanoma, leukemia (e.g., acute lymphocytic leukemia, chronic lymphocytic leukemia, acute myelogenous leukemia, chronic myelogenous leukemia, or hairy cell leukemia), lymphoma (e.g., non-Hodgkin's lymphoma. Hodgkin's lymphoma, B-cell lymphoma, or Burkitt's lymphoma), and multiple myeloma.

The term “lymphoproliferative disorders” refers to any disorders characterized by abnormal proliferation of lymphocytes into a monoclonal lymphocytosis. Non-limiting examples of different types of lymphoproliferative disorders suitable for treatment using the compositions and methods of the present invention besides leukemia as described above include Waldenström's macroglobulinemia, Wiskott-Aldrich syndrome, Langerhans cell histiocytosis, Lymphocyte-variant hypereosinophila, Pityriasis Lichenoides, Post-transplant lymphoproliferative disorder, Autoimmune lymphoproliferative syndrome, Lymphoid interstitial pneumonia, Epstein-Ban virus-associated lymphoproliferative diseases, Castleman disease, and X-linked lymphoproliferative disease.

The term “suppressor cells” refers to any lymphocytes that can suppress productive immune response such as antibody production or T cell proliferation through various mechanisms including cell-cell contact, cytokines and killing. Non-limiting examples of different types of suppressive immune cells suitable for treatment using the compositions and methods of the present invention include T regulatory cells, Tr1 cells, B regulatory cells, and myeloid-derived suppressor cells.

### DETAILED DESCRIPTION OF THE INVENTION

### Introduction

This study describes for the first time the involvement of Kaposi's sarcoma-associated herpesvirus (KSHV) latency associated nuclear antigen (LANA) in the host cellular ChAHP (CHD4, ADNP, HP1) protein complex as well as its implications in CHD4⁺ cell growth and viability. The segment of LANA that is responsible for binding to CHD4 and participating in the cellular function of the ChAHP protein complex has been identified and is proposed for use in the suppression of CHD4-expressing cells proliferation and therefore for therapeutic use in the treatment of disorders and diseases involving CHD4⁺ cells, such as CHD4⁺ cancers.

Peptides that Inhibit Proliferation and Survival of CHD4⁺ Cells Sequence

The present disclosure provides peptides derived from the Kaposi's sarcoma-associated herpesvirus (KSHV), in particular from a conserved 16-amino acid region of KSHV latency associated nuclear antigen (LANA), that is important for its interaction with CHD4, especially in the context of host cellular ChAHP complex. The 16-amino acid peptide from KSHV LANA, along with certain variants, are referred to herein as the VGN73 peptides, exemplary CHD4-binding peptides with the amino acid sequences set forth in SEQ ID NOs:1-8. By contacting CHD4⁺ cells with a CHD4-binding peptide such as the VGN73 peptide or a peptide based on/derived from VGN73 as described herein, including a fusion polypeptide comprising a CHD4-binding peptide and one or more heterologous peptide, suppression of proliferation of the CHD4⁺ cells can be achieved, and in some cases the cells are killed.

In particular embodiments, the peptide is at least 16 amino acids long and comprises (or consists of) the amino acid sequence of SEQ ID NO:1, or comprises a sequence identical to SEQ ID NO:1 at all but 1, 2, 3, 4, 5, or 6 positions. One example is SEQ ID NO:2. Other examples are SEQ ID NOs:3-8. Further examples are shown in various illustrations of this disclosure, including the drawings. In some embodiments, the amino acid sequence of the peptide is about 70%, 80%, 85%, 90%, 95% or more identical to SEQ ID NO:1. In particular embodiments, the peptide is identical to SEQ ID NO:1 at all amino acid positions except for one, two, three, or more at positions 2, 3, 4, 10, 11-15, optionally with the amino acids at positions 12-15 deleted. The CHD4-binding peptide retains its ability to bind CHD4 at an affinity at least 50%, 75%, 80%, or 90% or more compared to the binding affinity of the peptide of SEQ ID NO:1 to CHD4 under the same assay conditions. In some embodiments, the peptide is shorter than 100 amino acids. e.g., no more than 60, 70, or 80 amino acids, and in some embodiments, the peptide is longer than 16 amino acids, e.g., 17, 18, 19, 20, 25, 30, 40, or 50 or more amino acids. In other embodiments, the CHD4-binding peptide is shorter. e.g., about 11, 12, 13, 14, 15, or 16 amino acids (see SEQ ID NOs:2-8). In some embodiments, e.g., when the CHD4-binding peptide is present within a fusion protein with another moiety such as a cell-penetrating protein, the overall polypeptide comprising the peptide can be any length, e.g., about 25, 30, 35, 40, 45, 50, 55, 60, 65, 70, 75, 80, 85, 90, 95, 100, 150, 200, 250, 300, 350, 400, 450, 500 or more amino acids, or a length of about 20-25, 25-35, or 30-40 amino acids, 40-50, 50-60, 60-70, 70-80, 80-90, 90-100, 100-150, 150-200, 200-300, 300-400, 400-500, or more amino acids.

### Non-Standard Amino Acids

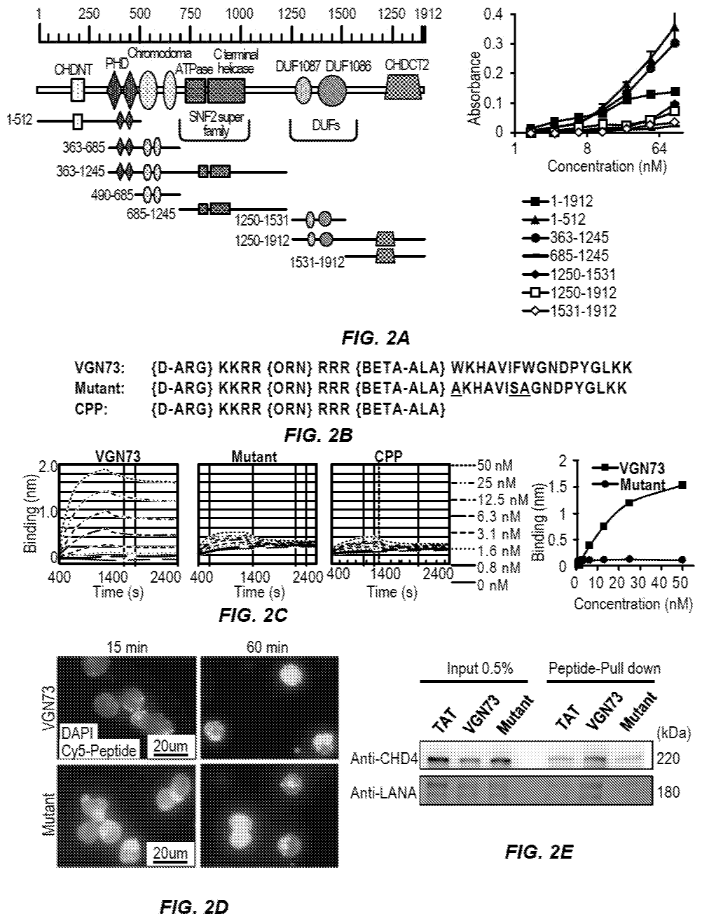

In some embodiments, the fusion polypeptide comprising the CHD4-binding peptide and one or more heterologous peptide(s) comprises one or more non-standard amino acids, such as D-amino acids, β-alanine, or ornithine, which may be located within the CHD4-binding peptide or one or more of the heterologous peptide(s), such as at the N-terminus of the CHD4-binding peptide, the heterologous peptide, or the fusion polypeptide. In some embodiments, one or more of the amino acids within the peptide is a D-amino acid. D-amino acids can be present at any position in the CHD4-binding peptide. e.g., at position 1, 2, 3, 4, 5, 6, 7, 8, 9, 10, 11, 12, 13, 14, 15, 16, or any combination of any of these positions. In particular embodiments, one or more D-amino acids are present at the C-terminal amino acid or acids of the peptide, e.g., at position 16, or at positions 15 and 16, or at positions 14, 15, and 16. In a particular embodiment, the two C-terminal amino acids of the peptide are D-amino acids. In some embodiments, one or more D-amino acids are present at the N-terminal amino acid or acids of the peptide, e.g., at position 1, or at positions 1 and 2, or at positions 1, 2, and 3. D-amino acids can also be incorporated into larger peptides or polypeptides, such as fusion proteins, comprising the 16-amino acid CHD4-binding peptide. For example, in some embodiments, polypeptides are made comprising a modified TAT peptide at the N-terminus linked via a peptide bond to the VGN73 peptide, in which the N-terminal amino acid of the fusion polypeptide is a D-amino acid. See, e.g., FIG. 2B and SEQ ID NOs:11-19.

In some embodiments, the peptide comprises other, non-standard amino acids, such as ornithine. β-alanine, and others. Such amino acids can be incorporated at any position within the 16-amino acid CHD4-binding peptide or in a larger polypeptide or fusion protein comprising the CHD4-binding peptide as well as one or more additional moieties such as an antibody (or an antigen-binding fragment thereof) or a cell penetrating peptide.

D-amino acids and other non-standard amino acids can be incorporated into the peptide using any suitable method. For example, they can be incorporated during chemical synthesis of the peptide using known methods, or during the production of recombinant peptides in cell-free systems using genetic code reprogramming (see, e.g., Katoh et al., Cell Chem Biol 24:46-64).

### Conjugates

In some embodiments, the CHD4-binding peptide of this invention is conjugated to a heterologous moiety. e.g., a moiety designed to allow easy isolation/identification of the peptide, to improve stability/bioavailability of the peptide, or to target the peptide to a specific cell type and/or facilitate entry into cells. The moiety can be attached to the peptide using a chemical linker, or, when the moiety is also a polypeptide, through a peptide bond, i.e., the two peptides or polypeptides are present in a single polypeptide chain as a fusion protein. In some cases, the linker is a cleavable peptide linker so as to allow easy separation of the peptide and its conjugation partner at the presence of an appropriate protease.

In some embodiments, the moiety is a cell penetrating peptide (CPP) (see, e.g., Patel et al. (2019) Scientific Reports 9: article no. 298). In particular embodiments, the CPP is a TAT peptide (GRKKRRQRRRPQ, designated as SEQ ID NO:9, derived from the transactivator of transcription (TAT) of HIV), or a variant or derivative thereof. In some embodiments, the CPP comprises one or more non-standard amino acids, e.g., a D-amino acid, β-alanine, and/or ornithine. In a particular embodiment, the TAT peptide comprises a D-amino acid, β-alanine, or ornithine, e.g., the sequence: d-Arg-KKRR-Omithine-RRR-β-alanine (SEQ ID NO:10). In particular embodiments, the TAT peptide (or other CPP) is present in a single polypeptide chain with the 16-amino acid CHD4-binding peptide or a variant. e.g., N-terminal to the CHD4-binding peptide, see, e.g., any one of SEQ ID NOs:11-18. In some embodiments, the TAT peptide is immediately N-terminal of the CHD4-binding peptide. In other embodiments, a linker and/or other elements are present between the TAT and CHD4-binding peptides.

In some embodiments, the conjugation partner of the CHD4-binding peptide is a therapeutic moiety, which may provide a therapeutic benefit similar to or different from that of the CHD4-binding peptide. The conjugation of the two partners not only can add a separate aspect of the conjugate in its therapeutic applications but also can enhance the efficacy of each partner alone. For instance, the presence of the therapeutic moiety can result in the increase of the anti-proliferation or anti-cancer efficacy of the CHD4-binding peptide by at least 20%, 30%, 40%, 50%, 60%, 70%, 80%, 90%, 100% or more, such as by at least 1.5, 2, 2.5, 3, 5, 10, 20, 25, 50, 80, 100, or 500, or 1000-fold. Further, the presence of the two partners in a close physical proximity can generate a synergistic effect of the two partners' combined therapeutic efficacy. For example, the resulting anti-cancer effect of the conjugate may represent an increase from the additive effect of the two partners by at least 50%, 100%, 150%, 200% or more, such as 3, 4, 5, 6, 7, 8, 9, 10-fold or more.

In some embodiments, the heterologous moiety serves to deliver the conjugate to a pre-determined target organ, tissue, or cell type. e.g., CHD4⁺ cancer cells, immune cells such as T or B cells, which would permit targeted treatment of malignancies such as breast cancer, lung cancer, and various types of lymphoproliferative disorders including leukemia and lymphoma (e.g., PEL and B-cell lymphoma).

### Antibod Conjugates

In some embodiments, the peptide of this invention is conjugated to an antibody or fragment thereof e.g., an antibody that binds specifically or preferentially to a cancer cell where CHD4 is expressed on the surface (i.e., a “CHD4-expressing cancer” or “CHD4⁺ cancer”) or to a CHD4⁺ lymphoid cell such as B or T cell involved in a lymphoproliferative disorder. In such embodiments, the antibody or antibody fragment can direct the peptide in vivo to CHD4-positive cancer cells where the peptide can suppress cancer cell proliferation and/or survival. In such embodiments, the antibody can be linked to the peptide by including both a single chain antibody and a peptide in a single polypeptide chain. In some embodiments, the peptide and the antibody are separated by a linker, e.g., a linker with a protease (for example, a furin protease or a matrix metalloprotease) cleavage site so as to liberate the peptide in the vicinity of the target cell. In other embodiments, an antibody or antibody fragment can be chemically linked to the peptide.

In addition to antibodies and antibody fragments, any molecule that binds specifically to a CHD4+ cancer cell or to a target CHD4+ lymphoid cell (such as B or T cell) can be linked to the peptides of this invention. For example, ligands to receptors on the surface of CHD4⁺ cancer cells can be used. Any molecule also present on the surface of CHD4⁺ cancer cells or lymphoid cells can be targeted. In some embodiments, the cancer is a primary effusion lymphoma (PEL) or multiple myeloma. In somer embodiments, the antigen recognized by the antibody is CD3, which allows the use of the CHD4-binding peptide conjugate for treating cancers such as CHD4⁺ T-cell lymphomas. In some embodiments, the antigen recognized by the antibody is EGFR, which allows the use of the CHD4-binding peptide conjugate for treating cancers such as CHD4⁺ breast cancer. In many cases, such antibody itself has anti-cancer efficacies, expecting synergistic effects. Combination of peptide conjugated and non-conjugated form to target same cell would further benefit activation of immune effects by ADCC and cancer cell growth inhibition or killing by virtue of CHD4-binding. Thus, the peptide may enhance the efficacy of therapeutic antibodies such as those targeting EGFR or VEGF, e.g., FDA-approved anti-cancer antibody drugs, including Bevacizumb, Ramucirumab that target VEGF, Cetuximab, and Trastuzumab targeting EGFR receptor as well as Herceptin for HER2. Often such antibodies are humanized in order to minimize any undesirable immune response. In some cases, the peptide of this invention is linked with a desired antibody (or an antigen-binding fragment thereof) and used by employing a clevable linker together with a protease such as matrix metalloprotease.

An exemplary immunoglobulin (antibody) structural unit comprises a tetramer. Each tetramer is composed of two identical pairs of polypeptide chains, each pair having one “light” chain (about 25 kDa) and one “heavy” chain (about 50-70 kDa). The N-terminus of each chain defines a variable region of about 100 to 110 or more amino acids primarily responsible for antigen recognition. Thus, the terms “variable heavy chain,” “V_{H}”, or “VH” refer to the variable region of an immunoglobulin heavy chain, including an Fv, scFv, dsFv or Fab; while the terms “variable light chain,” “V_{L}”, or “VL” refer to the variable region of an immunoglobulin light chain, including of an Fv, scFv, dsFv or Fab. Equivalent molecules include antigen binding proteins having the desired antigen specificity, derived, for example, by modifying an antibody fragment or by selection from a phage display library.

In some embodiments, the antibody is a monoclonal antibody. In some embodiments, the antibody is a polyclonal antibody. In some embodiments, the antibody is a chimeric antibody. In some embodiments, the antibody is a humanized antibody. In some embodiments, the antibody is a human antibody. In some embodiments, the antibody is an antigen-binding fragment, such as a F(ab′)2. Fab′, Fab, scFv, and the like. The term “antibody or antigen-binding fragment” can also encompass multi-specific and hybrid antibodies, with dual or multiple antigen or epitope specificities. In particular embodiments, the antibody is a single chain antibody.

In some embodiments, the antibody comprises a heavy chain sequence or a portion thereof, and/or a light chain sequence or a portion thereof, of an antibody sequence disclosed herein. In some embodiments, the antibody comprises one or more complementarity determining regions (CDRs) of an antibody as disclosed herein. In some embodiments, the antibody is a nanobody, or single-domain antibody (sdAb), comprising a single monomeric variable antibody domain. e.g., a single VHH domain.

### Other Elements

In addition to the CHD4-binding peptide and an optional moiety such as a CPP or antibody or antibody fragment, the peptides used in the present invention can comprise other elements such as linkers separating the different elements within a peptide, signal sequences, and nuclear localization sequences.

In some embodiments, two or more elements within a peptide of the invention are separated by a flexible linker. Suitable linkers for separating protein domains are known in the art, and can comprise, e.g., glycine and serine residues, for example, from 2-20 glycine and/or serine residues. In some embodiments, the linker can comprise protease cleavage sites, for example, serine protease cleavage sites, such that, if desired, the peptide can be separated from an heterologous peptide after being directed to a CHD4+ cell. In some embodiments, the peptide can comprise a nuclear localization signal, enabling the peptide to enter the nucleus where it can bind to a targeted cell. In some embodiments, the peptide comprises a cysteine residue at the C-terminus, to allow further chemical conjugation. In some embodiments, the peptide (or polypeptide) comprises a 16-amino acid CHD4-binding peptide of the invention, a humanized antibody targeting a specific cell or tissue type of interest, and a linker separating the antibody from the CHD4-binding peptide, wherein the linker comprises a protease cleavage site, and optionally a nuclear localization signal (NLS).

### Preparing Recombinant Peptides

The peptides of the invention, e.g., isolated CHD4-binding peptides and/or fusion proteins or polypeptides comprising CHD4-binding peptides as well as other moieties such as antibodies or CPPs, can be prepared in any number of ways, including through chemical peptide synthesis or through recombinant methods.

### Chemical Synthesis

The polypeptides of this invention may be synthesized by solid-phase peptide synthesis methods using procedures similar to those described by Merrifield et al., J. Am. Chem. Soc., 85:2149-2156 (1963); Barany and Merrifield. Solid-Phase Peptide Synthesis, in The Peptides: Analysis, Synthesis, Biology Gross and Meienhofer (eds.). Academic Press, N.Y., vol. 2, pp. 3-284 (1980); and Stewart et al., Solid Phase Peptide Synthesis 2nd ed., Pierce Chem. Co., Rockford, Ill. (1984). During synthesis, N-α-protected amino acids having protected side chains are added stepwise to a growing polypeptide chain linked by its C-terminal and to a solid support, i.e., polystyrene beads. The peptides are synthesized by linking an amino group of an N-α-deprotected amino acid to an α-carboxy group of an N-α-protected amino acid that has been activated by reacting it with a reagent such as dicyclohexylcarbodiimide. The attachment of a free amino group to the activated carboxyl leads to peptide bond formation. The most commonly used N-α-protecting groups include Boc, which is acid labile, and Fmoc, which is base labile.

### Recombinant Production

In some embodiments, the peptides or fusion proteins are produced recombinantly using standard molecular biology methods. For example, the nucleotide sequences coding the CHD4-binding peptide, and optionally an additional sequence such as a single chain antibody or a TAT peptide, can be synthesized using standard methods and cloned into a suitable expression vector, e.g., the His-tag expression vector pET30(a)+. Recombinant TnC and FABP can then be expressed in suitable cells, e.g., E. coli, and purified, and the protein concentrations and purities determined by. e.g., BCA assay and SDS-PAGE, respectively.

Basic texts disclosing general methods and techniques in the field of recombinant genetics include Sambrook and Russell, Molecular Cloning, A Laboratory Manual (3rd ed. 2001); Kriegler. Gene Transfer and Expression: A Laboratory Manual (1990); and Ausubel et al., eds., Current Protocols in Molecular Biology (1994).

For nucleic acids, sizes are given in either kilobases (kb) or base pairs (bp). These are estimates derived from agarose or acrylamide gel electrophoresis, from sequenced nucleic acids, or from published DNA sequences. For proteins, sizes are given in kilodaltons (kDa) or amino acid residue numbers. Proteins sizes are estimated from gel electrophoresis, from sequenced proteins, from derived amino acid sequences, or from published protein sequences.

Oligonucleotides that are not commercially available can be chemically synthesized. e.g., according to the solid phase phosphoramidite triester method first described by Beaucage & Caruthers, Tetrahedron Lett. 22: 1859-1862 (1981), using an automated synthesizer, as described in Van Devanter et. al., Nucleic Acids Res. 12: 6159-6168 (1984). Purification of oligonucleotides is performed using any art-recognized strategy, e.g., native acrylamide gel electrophoresis or anion-exchange HPLC as described in Pearson & Reanier, J. Chrom. 255: 137-149 (1983).

The sequence of a polynucleotide encoding a peptide of this invention can be verified after cloning or subcloning using, e.g., the chain termination method for sequencing double-stranded templates of Wallace et al., Gene 16: 21-26 (1981).

Polynucleotide sequences encoding the peptides of this invention can be determined based on their amino acid sequences. They can be isolated, e.g., from a KSHV genomic library or can be synthesized by a commercial supplier. Nucleic acid sequences encoding the peptides of this invention can be isolated using standard cloning techniques such as polymerase chain reaction (PCR). Most commonly used techniques for this purpose are described in standard texts. e.g., Sambrook and Russell, supra.

Based on sequence homology, degenerate oligonucleotides can be designed as primer sets and PCR can be performed under suitable conditions (see. e.g., White et al., PCR Protocols: Current Methods and Applications, 1993: Griffin and Griffin. PCR Technology, CRC Press Inc. 1994) to amplify a segment of nucleotide sequence from a cDNA or genomic library. Using the amplified segment as a probe, a longer length nucleic acid encoding a peptide of this invention is obtained.

Upon acquiring a nucleic acid sequence encoding a peptide of this invention, the coding sequence can be modified as appropriate (e.g., adding a coding sequence for a heterologous tag, such as an affinity tag, for example. 6×His tag or GST tag) and then be subcloned into a vector, for instance, an expression vector, so that a recombinant peptide can be produced from the resulting construct, for example, after transfection and culturing host cells under conditions permitting recombinant protein expression directed by a promoter operably linked to the coding sequence.

In some embodiments, the polynucleotide sequence encoding a peptide of the invention can be further altered to coincide with the preferred codon usage of a particular host. For example, the preferred codon usage of one strain of bacterial cells can be used to derive a polynucleotide that encodes a peptide of this invention and includes the codons favored by this strain. The frequency of preferred codon usage exhibited by a host cell can be calculated by averaging frequency of preferred codon usage in a large number of genes expressed by the host cell (e.g., calculation service is available from web site of the Kazusa DNA Research Institute. Japan). This analysis is preferably limited to genes that are highly expressed by the host cell.

To obtain high level expression of a nucleic acid encoding a peptide of the present invention, a polynucleotide encoding the polypeptide can be subcloned into an expression vector that contains a strong promoter (typically heterologous) to direct transcription, a transcription/translation terminator and a ribosome binding site for translational initiation. Suitable bacterial promoters are well known in the art and described. e.g., in Sambrook and Russell, supra, and Ausubel et al., supra. Bacterial expression systems for expressing a recombinant polypeptide are available in, e.g., E. coli, Bacillus sp., Salmonella, and Caulobacter. Kits for such expression systems are commercially available. Eukaryotic expression systems for mammalian cells, yeast, and insect cells are well known in the art and are also commercially available. In one embodiment, the eukaryotic expression vector is an adenoviral vector, an adeno-associated vector, or a retroviral vector.

The promoter used to direct expression of a heterologous nucleic acid depends on the particular application. The promoter is optionally positioned about the same distance from the heterologous transcription start site as it is from the transcription start site in its natural setting. As is known in the art, however, some variation in this distance can be accommodated without loss of promoter function. In one embodiment, the promoter is an IPTG-inducible promoter.

In addition to the promoter, the expression vector typically includes a transcription unit or expression cassette that contains all the additional elements required for the expression of the peptide in host cells. A typical expression cassette thus contains a promoter operably linked to the coding sequence and signals required for efficient polyadenylation of the transcript, ribosome binding sites, and translation termination. The nucleic acid sequence encoding the peptide is typically linked to a cleavable signal peptide sequence to promote secretion of the recombinant polypeptide by the transformed cell. Such signal peptides include, among others, the signal peptides from tissue plasminogen activator, insulin, and neuron growth factor, and juvenile hormone esterase of Heliothis virescens. Additional elements of the cassette may include enhancers and, if genomic DNA is used as the structural gene, introns with functional splice donor and acceptor sites.

In addition to a promoter sequence, the expression cassette should also contain a transcription termination region downstream of the structural gene to provide for efficient termination. The termination region may be obtained from the same gene as the promoter sequence or may be obtained from different genes.

The particular expression vector used to transport the genetic information into the cell is not particularly critical. Any of the conventional vectors used for expression in eukaryotic or prokaryotic cells may be used. Standard bacterial expression vectors include plasmids such as pBR322 based plasmids, pSKF, pET23D, pET30(a)+, and fusion expression systems such as GST and LacZ. Epitope tags can also be added to recombinant proteins to provide convenient methods of isolation, e.g., c-myc.

Expression vectors containing regulatory elements from eukaryotic viruses are typically used in eukaryotic expression vectors, e.g., SV40 vectors, papilloma virus vectors, and vectors derived from Epstein-Barr virus. Other exemplary eukaryotic vectors include pMSG, pAV009/A⁺, pMTO10/A⁺, pMAMneo-5, baculovirus pDSVE, and any other vector allowing expression of proteins under the direction of the SV40 early promoter, SV40 later promoter, metallothionein promoter, murine mammary tumor virus promoter. Rous sarcoma virus promoter, polyhedrin promoter, or other promoters shown effective for expression in eukaryotic cells.

Some expression systems have markers that provide gene amplification such as thymidine kinase, hygromycin B phosphotransferase, and dihydrofolate reductase. Alternatively, high yield expression systems not involving gene amplification are also suitable, such as a baculovirus vector in insect cells, with a polynucleotide sequence encoding the peptide under the direction of the polyhedrin promoter or other strong baculovirus promoters.

The elements that are typically included in expression vectors also include a replicon that functions in E. coli, a gene encoding a protein that provides antibiotic resistance to permit selection of bacteria that harbor recombinant plasmids, and unique restriction sites in nonessential regions of the plasmid to allow insertion of eukaryotic sequences. The particular antibiotic resistance gene chosen is not critical, any of the many resistance genes known in the art are suitable. The prokaryotic sequences are optionally chosen such that they do not interfere with the replication of the DNA in eukaryotic cells, if necessary. Similar to antibiotic resistance selection markers, metabolic selection markers based on known metabolic pathways may also be used as a means for selecting transformed host cells.

When periplasmic expression of a recombinant protein (e.g., a CHD4-binding peptide or fusion protein of the present invention) is desired, the expression vector further comprises a sequence encoding a secretion signal, such as the E. coli OppA (Periplasmic Oligopeptide Binding Protein) secretion signal or a modified version thereof, which is directly connected to 5′ of the coding sequence of the protein to be expressed. This signal sequence directs the recombinant protein produced in cytoplasm through the cell membrane into the periplasmic space. The expression vector may further comprise a coding sequence for signal peptidase 1, which is capable of enzymatically cleaving the signal sequence when the recombinant protein is entering the periplasmic space. More detailed description for periplasmic production of a recombinant protein can be found in. e.g., Gray et al., Gene 39: 247-254 (1985), U.S. Pat. Nos. 6,160,089 and 6,436,674.

### Transfection

Standard transfection methods are used to produce bacterial, mammalian, yeast, insect, or plant cell lines that express large quantities of a recombinant polypeptide, which are then purified using standard techniques (see, e.g., Colley et al., J. Biol. Chem. 264: 17619-17622 (1989); Guide to Protein Purification, in Methods in Enzymology, vol. 182 (Deutscher, ed., 1990)). Transformation of eukaryotic and prokaryotic cells are performed according to standard techniques (see, e.g., Morrison, J. Bact. 132: 349-351 (1977); Clark-Curtiss & Curtiss, Methods in Enzymology 101: 347-362 (Wu et al., eds. 1983).

Any of the well-known procedures for introducing foreign nucleotide sequences into host cells may be used. These include the use of calcium phosphate transfection, polybrene, protoplast fusion, electroporation, liposomes, microinjection, plasma vectors, viral vectors and any of the other well-known methods for introducing cloned genomic DNA, cDNA, synthetic DNA, or other foreign genetic material into a host cell (see, e.g., Sambrook and Russell, supra). It is only necessary that the particular genetic engineering procedure used be capable of successfully introducing at least one gene into the host cell capable of expressing the recombinant polypeptide.

### Detection of Expression in Host Cells

After the expression vector is introduced into appropriate host cells, the transfected cells are cultured under conditions favoring expression of the peptide. The cells are then screened for the expression of the recombinant polypeptide, which is subsequently recovered from the culture using standard techniques (see, e.g., Scopes, Protein Purification: Principles and Practice (1982); U.S. Pat. No. 4,673,641; Ausubel et al., supra; and Sambrook and Russell, supra).

Several general methods for screening gene expression are well known among those skilled in the art. First, gene expression can be detected at the nucleic acid level. A variety of methods of specific DNA and RNA measurement using nucleic acid hybridization techniques are commonly used (e.g., Sambrook and Russell, supra). Some methods involve an electrophoretic separation (e.g., Southern blot for detecting DNA and Northern blot for detecting RNA), but detection of DNA or RNA can be carried out without electrophoresis as well (such as by dot blot). The presence of nucleic acid encoding a peptide in transfected cells can also be detected by PCR or RT-PCR using sequence-specific primers.

Second, gene expression can be detected at the polypeptide level. Various immunological assays are routinely used by those skilled in the art to measure the level of a gene product, particularly using polyclonal or monoclonal antibodies that react specifically with a peptide of this invention (e.g., Harlow and Lane, Antibodies, A Laboratory Manual, Chapter 14, Cold Spring Harbor, 1988; Kohler and Milstein, Nature, 256: 495-497 (1975)). Such techniques require antibody preparation by selecting antibodies with high specificity against the peptide. The methods of raising polyclonal and monoclonal antibodies are well established and their descriptions can be found in the literature, see, e.g., Harlow and Lane, supra: Kohler and Milstein, Eur. J. Immunol., 6: 511-519 (1976).

### Purification of Recombinantly Produced Peptides

Once the expression of a recombinant peptide of this invention in transfected host cells is confirmed, the host cells are then cultured in an appropriate scale for the purpose of purifying the recombinant polypeptide.

When the peptides of the present invention are produced recombinantly by transformed bacteria in large amounts, typically after promoter induction, although expression can be constitutive, the polypeptides may form insoluble aggregates. There are several protocols that are suitable for purification of protein inclusion bodies. For example, purification of aggregate proteins (hereinafter referred to as inclusion bodies) typically involves the extraction, separation and/or purification of inclusion bodies by disruption of bacterial cells. e.g., by incubation in a buffer of about 100-150 μg/ml lysozyme and 0.1% Nonidet P40, a non-ionic detergent. The cell suspension can be ground using a Polytron grinder (Brinkman Instruments, Westbury, NY). Alternatively, the cells can be sonicated on ice. Alternate methods of lysing bacteria are described in Ausubel et al, and Sambrook and Russell, both supra, and will be apparent to those of skill in the art.

The cell suspension is generally centrifuged and the pellet containing the inclusion bodies resuspended in buffer which does not dissolve but washes the inclusion bodies, e.g., 20 mM Tris-HCl (pH 7.2). 1 mM EDTA. 150 mM NaCl and 2% Triton-X 100, a non-ionic detergent. It may be necessary to repeat the wash step to remove as much cellular debris as possible. The remaining pellet of inclusion bodies may be resuspended in an appropriate buffer (e.g., 20 mM sodium phosphate. pH 6.8, 150 mM NaCl). Other appropriate buffers will be apparent to those of skill in the art.

Following the washing step, the inclusion bodies are solubilized by the addition of a solvent that is both a strong hydrogen acceptor and a strong hydrogen donor (or a combination of solvents each having one of these properties). The proteins that formed the inclusion bodies may then be renatured by dilution or dialysis with a compatible buffer. Suitable solvents include, but are not limited to, urea (from about 4 M to about 8 M), formamide (at least about 80%, volume/volume basis), and guanidine hydrochloride (from about 4 M to about 8 M). Some solvents that are capable of solubilizing aggregate-forming proteins, such as SDS (sodium dodecyl sulfate) and 70% formic acid, may be inappropriate for use in this procedure due to the possibility of irreversible denaturation of the proteins, accompanied by a lack of immunogenicity and/or activity. Although guanidine hydrochloride and similar agents are denaturants, this denaturation is not irreversible and renaturation may occur upon removal (by dialysis, for example) or dilution of the denaturant, allowing re-formation of the immunologically and/or biologically active protein of interest. After solubilization, the protein can be separated from other bacterial proteins by standard separation techniques. For further description of purifying recombinant polypeptides from bacterial inclusion body, see, e.g., Patra et al., Protein Expression and Purification 18: 182-190 (2000).

Alternatively, it is possible to purify recombinant polypeptides from bacterial periplasm. Where the recombinant protein is exported into the periplasm of the bacteria, the periplasmic fraction of the bacteria can be isolated by cold osmotic shock in addition to other methods known to those of skill in the art (see e.g., Ausubel et al., supra). To isolate recombinant proteins from the periplasm, the bacterial cells are centrifuged to form a pellet. The pellet is resuspended in a buffer containing 20% sucrose. To lyse the cells, the bacteria are centrifuged and the pellet is resuspended in ice-cold 5 mM MgSO₄ and kept in an ice bath for approximately 10 minutes. The cell suspension is centrifuged and the supernatant decanted and saved. The recombinant proteins present in the supernatant can be separated from the host proteins by standard separation techniques well known to those of skill in the art.

### Protein Separation Techniques for Purification

When a recombinant polypeptide is expressed in host cells in a soluble form, its purification can follow a standard protein purification procedure as described herein. Such standard purification procedures are also suitable for purifying a polypeptide obtained from chemical synthesis.

### Solubility Fractionation

Often as an initial step, and if the protein mixture is complex, an initial salt fractionation can separate many of the unwanted host cell proteins (or proteins derived from the cell culture media) from the recombinant protein of interest. The preferred salt is ammonium sulfate. Ammonium sulfate precipitates proteins by effectively reducing the amount of water in the protein mixture. Proteins then precipitate on the basis of their solubility. The more hydrophobic a protein is, the more likely it is to precipitate at lower ammonium sulfate concentrations. A typical protocol is to add saturated ammonium sulfate to a protein solution so that the resultant ammonium sulfate concentration is between 20-30%. This will precipitate the most hydrophobic proteins. The precipitate is discarded (unless the protein of interest is hydrophobic) and ammonium sulfate is added to the surpenatant to a concentration known to precipitate the protein of interest. The precipitate is then solubilized in buffer and the excess salt removed if necessary, through either dialysis or diafiltration. Other methods that rely on solubility of proteins, such as cold ethanol precipitation, are well known to those of skill in the art and can be used to fractionate complex protein mixtures.

### Size Differential Filtration

Based on a calculated molecular weight, a protein of greater and lesser size can be isolated using ultrafiltration through membranes of different pore sizes (for example, Amicon or Millipore membranes). As a first step, the protein mixture is ultrafiltered through a membrane with a pore size that has a lower molecular weight cut-off than the molecular weight of a protein of interest. e.g., CHD4-binding peptide. The retentate of the ultrafiltration is then ultrafiltered against a membrane with a molecular cut off greater than the molecular weight of the protein of interest. The recombinant protein will pass through the membrane into the filtrate. The filtrate can then be chromatographed as described below.

### Column Chromatography

The proteins of interest (such as a CHD4-binding peptide or a fusion polypeptide thereof as described herein) can also be separated from other proteins on the basis of their size, net surface charge, hydrophobicity, or affinity for ligands. In addition, antibodies raised against the peptide can be conjugated to column matrices and the corresponding peptide immuno-purified. All of these methods are well known in the art. It will be apparent to one of skill that chromatographic techniques can be performed at any scale and using equipment from many different manufacturers (e.g., Pharmacia Biotech).

### Assessing CHD4-Binding and Inhibition of CHD4-Expressing Cells

A CHD4-binding peptide of this invention or a fusion proteion thereof has the utility of treating a disease such as a cancer, especially a lymphoproliferative disease, by targeting CHD4-expressing cells. Such function of a CHD4-binding peptide, especially one having an amino acid sequence modified from the exemplary sequence of SEQ ID NO:1 or 2, may be tested and verified by any one of the methods known in the pertinent technical field or described herein. Two aspects can be tested to assess a peptide's potential function as a CHD4-binding peptide: first, it can be tested for its ability to bind the CHD4 protein. Second, it can be tested for its ability to inhibit proliferation of CHD4⁺ cells or to kill the cells.

For protein binding assays, a peptide may be tested using routine techniques for its ability to bind the CHD4 protein, either in a cell-free assay format or in a cell-based assay format. In a cell-free format, a peptide is placed together with the CHD4 protein under suitable conditions for protein-protein interaction to take place. Typically, a known CHD4-binding peptide (e.g., SEQ ID NO:1 or 2) is used as a positive control as well as a comparison basis in such an assay. A peptide, such as a variant containing one or more amino acid residue of SEQ ID NO:1 or 2 modified by addition, deletion, and/or substitution, that retains at least about 25%, 50%, or 75% of the original ability to bind CHD4 in comparison to SEQ ID NO:1 or 2 under the same assay conditions is presumed as a CHD4-binding peptide and may be further tested for its ability to inhibit CHD4⁺ cell growth or survival. In a cell-based format, a peptide is tested for its ability to bind cell surface expressed CHD4 protein. In such assays, any CHD4-expressing cells can be used. For example, CHD4⁺ primary effusion lymphoma (PEL) cells can be used, such as BC-1, BC-3, BCBL-1, or BJAB cells. Similar to cell-free assays, a variant peptide retaining at least about 25%, 50%, or 75% of the original ability to bind cell surface CHD4 in comparison to SEQ ID NO:1 under the same assay conditions is presumed as a CHD4-binding peptide and may be further tested for its ability to inhibit CHD4⁺ cell growth or survival. Detection of protein binding using routine techniques such as Western blot assays, two-dimensional gel electrophoresis, and quantitative mass spectrometry that are known to those skilled in the art. Additional immunoassays, including but not limited to enzyme immunoassays (EIA), such as enzyme multiplied immunoassay technique (EMIT), enzyme-linked immunosorbent assay (ELISA). IgM antibody capture ELISA (MAC ELISA), and microparticle enzyme immunoassay (MEIA): capillary electrophoresis immunoassays (CEIA); radioimmunoassays (RIA); immunoradiometric assays (IRMA); immunofluorescence (IF); fluorescence polarization immunoassays (FPIA); and chemiluminescence assays (CL) may also be used in these assays.

Cell proliferation/survival assays measure the number of cultured CHD4⁺ cells upon exposure to a presumptive CHD4-binding peptide to confirm the peptide's ability to suppress the cellular proliferation and survival. In some embodiments, the peptide is assessed by determining its ability to inhibit CHD4-expressing cancer cell growth in vitro. For example, the growth and/or survival of PEL cells such as BC3 cells in culture can be measured, e.g., using an MTS or TUNNEL assay. In another example, the growth of cells such as CHD4⁺ malignant lymphoma cells such as NU-DUL-1 can be assessed, e.g., their growth in soft agar. The peptide can also be assessed in vivo using animal models. e.g., in tumor growth assays in xenograft models such as PEL cell xenograft models, by monitoring change in the tumor mass. Typically, a known CHD4-binding peptide such as SEQ ID NO:1 or 2 is used in these assays as a positive control as well as a comparison basis. A test peptide retaining at least about 25%, 50%, or 75% of the original ability to suppress CHD4⁺ cell growth or survival in comparison to SEQ ID NO:1 or 2 under the same conditions is deemed a CHD4-binding peptide suitable for use in accordance with the present invention.

The CHD4-binding peptides, including isolated CHD4-binding peptides as well as larger peptides or polypeptides comprising CHD4-binding peptides plus other elements, can also be assessed for their pharmacokinetic and/or pharmacodynamic properties. In some embodiments, the stability of the peptides and/or fusion proteins is assessed in vivo. In some embodiments, the localization of the peptides and/or fusion proteins is assessed in vivo, including in the vicinity of cells targeted by virtue of expressing CHD4 on its cell surface or by an antibody (or an antigen-binding fragment thereof) within a fusion protein.

In some embodiments, the polynucleotide sequence encoding the CHD4-binding peptide of SEQ ID NO:1 or 2 or a variant derived from SEQ ID NO:1 or 2 (e.g., any one of SEQ ID NOs:3-8) or a fusion peptide thereof is delivered to the intended recipient by using a viral vector. Suitable viral vectors can be derived from the genome of a human or animal adenovirus, vaccinia virus, herpes virus, adeno-associated virus (AAV), minute virus of mice (MVM), and retroviruses (including but not limited to Rous sarcoma virus and lentivirus), Maloney Murine Leukemia Virus (MoMLV), and the like. Typically, the coding sequence of interest (e.g., one encoding for SEQ ID NO:1 or 2 or its derivative or a fusion protein thereof as described herein) are inserted into such vectors to allow packaging of the gene construct, typically with accompanying viral DNA, followed by infection of a sensitive host cell and expression of the coding sequence of interest. In other embodiments, cells comprising the CHD4-binding peptide of SEQ ID NO:1 or 2 or a variant derived from SEQ ID NO:1 or 2 (e.g., any one of SEQ ID NOs:3-8) or a fusion peptide thereof, the polynucleotide sequence encoding the peptide or fusion peptide, or a vector such as an expression cassette comprising the polynucleotide coding sequence are delivered to the intended recipient in a pharmaceutical composition described herein.

### Dosage and Administration

### Subjects

The subject can be any subject. e.g., a human or another mammal, with a condition linked to excess CHD4⁺ cell proliferation. In particular embodiments, the subject has a CHD4-positive cancer, such as primary effusion lymphoma (PEL) or multiple myeloma. In some embodiments, the subject has a lymphoproliferative disorder involving inappropriately activated CHD4-positive lymphoid cells such as B or T cells. In some embodiments, the subject is a human. In some embodiments, the subject is an adult. In some embodiments, the subject is a child (e.g., a child aged 18 or younger). In some embodiments, the subject is female (e.g., an adult female). In some embodiments, the subject is male (e.g., an adult male).

### Pharmaceutical Compositions

The present disclosure provides compositions comprising (or consisting essentially of) an isolated and/or purified CHD4-binding peptide, which is capable of binding to CHD4 protein as well as ChAHP complex component peptides and thus inhibiting proliferation of CHD4⁺ cells, and one or more pharmaceutically acceptable carrier. As such, the present disclosure provides pharmaceutical compositions for inhibiting CHD4⁺ cells of a subject, for killing CHD4-positive cancer cells such as malignant CHD4⁺ lymphoid cells (e.g., B or T cells) in a subject, and for treating a CHD4⁺ cancer or lymphoproliferative disorder in a subject.

The pharmaceutical compositions of the present invention may comprise one or more pharmaceutically acceptable carrier. In certain aspects, pharmaceutically acceptable carriers are determined in part by the particular composition being administered, as well as by the particular method used to administer the composition. Accordingly, there is a wide variety of suitable formulations of pharmaceutical compositions of the present invention (see, e.g., REMINGTON'S PHARMACEUTICAL SCIENCES. 18TH ED., Mack Publishing Co., Easton, PA (1990)).

The pharmaceutical compositions will often further comprise one or more buffers (e.g., neutral buffered saline or phosphate buffered saline), carbohydrates (e.g., glucose, mannose, sucrose or dextrans), mannitol, proteins, polypeptides or amino acids such as glycine, antioxidants (e.g., ascorbic acid, sodium metabisulfite, butylated hydroxytoluene, butylated hydroxyanisole, etc.), bacteriostats, chelating agents such as EDTA or glutathione, solutes that render the formulation isotonic, hypotonic or weakly hypertonic with the blood of a recipient, suspending agents, thickening agents, preservatives, flavoring agents, sweetening agents, and coloring compounds as appropriate.

The pharmaceutical compositions of the invention are administered in a manner compatible with the dosage formulation, and in such amount as will be therapeutically or prophylactically effective. The quantity to be administered depends on a variety of factors including, e.g., the age, body weight, physical activity, hereditary characteristics, general health, sex, and diet of the individual, the condition or disease to be treated or prevented, and the stage or severity of the condition or disease. In certain embodiments, the size of the dose may also be determined by the existence, nature, and extent of any adverse side effects that accompany the administration of a therapeutic or prophylactic agent(s) in a particular individual. Other factors that can influence the specific dose level and frequency of dosage for any particular patient include the activity of the specific compound employed, the metabolic stability and length of action of that compound, the mode and time of administration, and the rate of excretion.

Generally, for administering the compound (e.g., a conjugate comprising a CHD4-binding peptide or a variant thereof and a heterologous moiety or a nucleic acid encoding a fusion protein comprising a CHD4-binding peptide or a variant thereof and a heterologous polypeptide or in a liposome form) for therapeutic or prophylactic purposes, the compound is given at a therapeutically or prophylactically effective dose. In particular, an effective amount of a pharmaceutical composition of the invention is an amount that is sufficient to eliminate the presence of CHD4 cells in the subject, or to slow, prevent, or reverse the growth of CHD4-expressing cancer cells in the subject.

In certain embodiments, the dose may take the form of solid, semi-solid, lyophilized powder, or liquid dosage forms, such as, for example, tablets, pills, pellets, capsules, powders, solutions, suspensions, emulsions, suppositories, retention enemas, creams, ointments, lotions, gels, aerosols, foams, or the like, preferably in unit dosage forms suitable for simple administration of precise dosages.

As used herein, the term “unit dosage form” refers to physically discrete units suitable as unitary dosages for humans and other mammals (e.g., an ampoule), each unit containing a predetermined quantity of a therapeutic or prophylactic agent calculated to produce the desired onset, tolerability, and/or therapeutic or prophylactic effects, in association with a suitable pharmaceutical excipient. In addition, more concentrated dosage forms may be prepared, from which the more dilute unit dosage forms may then be produced. The more concentrated dosage forms thus will contain substantially more than, e.g., at least 1, 2, 3, 4, 5, 6, 7, 8, 9, 10, or more times the amount of the therapeutic or prophylactic compound.

Methods for preparing such dosage forms are known to those skilled in the art (see. e.g., REMINGTON'S PHARMACEUTICAL SCIENCES, supra). The dosage forms typically include a conventional pharmaceutical carrier or excipient and may additionally include other medicinal agents, carriers, adjuvants, diluents, tissue permeation enhancers, solubilizers, and the like. Appropriate excipients can be tailored to the particular dosage form and route of administration by methods well known in the art (see, e.g., REMINGTON'S PHARMACEUTICAL SCIENCES, supra).

In some embodiments, the composition comprising the CHD4-binding peptide of this invention or a nucleic acid encoding the CHD4-binding peptide is formulated as a composition of nanoparticles, for example, in the form of lipid nanoparticles (LNP) comprising the peptide or nucleic acid. One or more types of nanoparticles, as well as other ingredients (such as lipids), may be used in the formulation. For example, the LNP may comprise a cationic lipid, a neutral lipid, a steroid, a polymer conjugated lipid, and the peptide or nucleic acid. In some cases, the LNP may further comprise at least one lipid or lipid-like material other than a cationic or cationically ionizable lipid or lipid-like material, at least one polymer other than a cationic polymer, or a mixture thereof. In some embodiments, the ratio of CHD4-binding peptide or nucleic acid to total lipid (N/P) is between 5 and 10 such as about 6 or about 7. Nanoparticles of this invention may have an average diameter ranging from about 30 nm to about 1000 nm, from about 50 nm to about 800 nm, from about 70 nm to about 600 am, from about 90 nm to about 400 nm, or from about 100 nm to about 300 mu. In some embodiments, the nanoparticles have an average diameter of about 50 nm, about 100 nm, or about 200 nm. The nanoparticles may exhibit a polydispersity index less than about 0.5, less than about 0.4, less than about 0.3, or about 0.2 or less. By way of example, the nanoparticles can exhibit a polydispersity index in a range of about 0.1 to about 0.3 or about 0.2 to about 0.3.

The preferred mode of administration of such peptide/nucleic acid-encapsulated LNP compositions is intertumoral or intravenous administration, more preferably in aqueous cryoprotectant buffer for intertumoral or intravenous administration. The composition is often a preservative-free, sterile dispersion of peptide or nucleic acid formulated in LNP in aqueous cryoprotectant buffer for intertumoral or intravenous administration.

### Administration

In some embodiments, prevention and/or treatment includes administering compositions of the present invention directly to a subject. As a non-limiting example, pharmaceutical compositions of the present invention (e.g., containing a CHD4-binding peptide conjugate of the invention, a nucleic acid encoding a fusion protein comprising a CHD4-binding peptide, or an engineered cell comprising such a nucleic acid including expression cassette encoding a fusion protein comprising a CHD4-binding peptide as described herein plus a pharmaceutically acceptable carrier) can be delivered directly to a subject (e.g., by local application or systemic administration).

Compositions of the present invention may be administered as a single dose or as multiple doses, for example two doses administered at an interval of about one month, about two months, about three months, about six months, or about 12 months. Other suitable dosage schedules can be determined by a medical practitioner.

In some embodiments, additional compounds or medications can be co-administered to the subject. Such compounds or medications can be co-administered for the purpose of alleviating signs or symptoms of the disease being treated, reducing side effects caused by treatment with the peptide, reducing cancer growth or killing cancer cells through a different mechanism, etc.

The pharmaceutical compositions of the invention can be administered locally or systemically to the subject, e.g., intraperitoneally, intramuscularly, intra-arterially, orally, intravenously, intracranially, intrathecally, intraspinally, intralesionally, intranasally, subcutaneously, intracerebroventricularly, topically, and/or by inhalation nasally.

### Kits

In another aspect, kits are provided herein. In some embodiments, the kit comprises a CHD4-inhibiting peptide, such as a VGN73 peptide and/or fusion protein of the invention. In some embodiments, the kit is for reducing, arresting, or preventing the proliferation of CHD4⁺ cells or reversing the presence of CHD4⁺ cells by causing the apoptosis of the cells, which include CHD4⁺ cancer cells in a subject's body. In some embodiments, the kit is for preventing or treating a disease. e.g., a cancer such as PEL.

Kits of the present invention can be packaged in a way that allows for safe or convenient storage or use (e.g., in a box or other container having a lid). Typically, kits of the present invention include one or more containers, each container storing a particular kit component such as a first composition comprising a CHD4-binding peptide or a fusion protein thereof, a second composition comprising another, different anti-cancer therapeutic agent, and so on. The choice of container will depend on the particular form of its contents, e.g., a kit component that is in liquid form, powder form, etc. Furthermore, containers can be made of materials that are designed to maximize the shelf-life of the kit components. As a non-limiting example, kit components that are light-sensitive can be stored in containers that are opaque.

In some embodiments, the kit contains one or more elements, e.g. syringe, useful for administering compositions (i.e., a pharmaceutical composition of the invention) to a subject. In yet other embodiments, the kit further comprises instructions for use, e.g., containing directions (i.e., protocols) for the practice of the methods of this invention (e.g., instructions for using the kit for inhibiting growth of CHD4⁺ cells or for treating a subject suffering from a CHD4⁺ cancer such as a lymphoproliferative disorder). While the instructional materials typically comprise written or printed materials, they are not limited to such. Any medium capable of storing such instructions and communicating them to an end user is contemplated by this invention. Such media include, but are not limited to electronic storage media (e.g., magnetic discs, tapes, cartridges, chips), optical media (e.g., CD ROM), and the like. Such media may include addresses to internet sites that provide such instructional materials.

### EXAMPLES

The following examples are provided by way of illustration only and not by way of limitation. Those of skill in the art will readily recognize a variety of non-critical parameters that could be changed or modified to yield essentially the same or similar results.

Kaposi's sarcoma-associated herpesvirus (KSHV) establishes a latent infection, in which most viral genes are silenced except a few latent proteins such as latency-associated nuclear antigen (LANA). In latent viral chromatin, viral genes are poised to be transcribed, and KSHV LANA plays a major role in maintaining such transcription status. In the poised chromatins, LANA recruits cellular CHD4 (Chromodomain Helicase DNA binding protein 4) and suppresses inducible viral gene promoters. The CHD4 is known to regulate cell differentiation by restricting enhancer-promoter interactions and its mutation or overexpression deregulates the host cell transcription program and therefore associates with tumorigenesis. Here, we identified a putative CHD4 inhibitor from the LANA amino acid sequence from the LANA-CHD4 interaction surface. The small peptide interacts with CHD4 at its PHD domain with 14 nM K_{D} (dissociation constant). The introduction of the peptide into the primary effusion lymphomas induces caspase-mediated CHD4 cleavage and subsequently triggered cell apoptosis and autophagy. A series of MTT assays demonstrated that the peptide preferentially killed lymphoma and leukemia cell lines at low micromolar concentrations, while peripheral mononuclear cells or adhesion cell lines were found to be more resistant to the peptide treatment. A monocyte cell differentiation model demonstrated that pre-treatment with the peptide substantially enhanced transitioning into macrophage, and globally altered the repertories of phorbol myristate acetate target genes in U937 cells. Finally, the PEL xenograft mouse model inhibited tumor growth without measurable side effects, and PEL cells isolated from xenograft tumors showed reduced CHD4 and LANA expression with differentiated marker expression. These results indicate that the peptide isolated from the KSHV LANA sequence is biologically active and functions as a CHD4 inhibitor by releasing CHD4-mediated restriction at gene regulatory regions.

### Introduction

Kaposi's sarcoma-associated herpesvirus (KSHV), also known as human herpesvirus 8 (HHV-8), is one of eight human herpesviruses^{1, 2}. KSHV is a causative agent of Kaposi's Sarcoma (KS)^{1,2}, two lymphoproliferative diseases, primary effusion lymphoma (PEL)^{3,4}, and multicentric Castleman's disease (MCD)^{5,6}. KSHV is also responsible for an interleukin-6-related disease called KSHV inflammatory cytokine syndrome (KICS)^{7,8}. Like other herpesviruses. KSHV's life cycle consists of lifelong latent infection and transient lytic replication, in which viral progenies are produced^{9, 10}. In latency, the expression of most viral genes are silenced and only a few selected genomic segments are actively transcribed¹¹. Among the genes expressed in a latent phase, ORF73 encodes the Latency Associated Nuclear Antigen (LANA) protein, and LANA functions to maintain latent episomes in infected cells¹². LANA tethers KSHV episomes by directly binding to terminal repeat (TR) sequences in the KSHV genomic DNA through its C-terminal DNA binding domain, and docks onto the host chromosome through its N-terminal histone binding domain, which enables the viral genomic DNA to hitch a ride on the host chromosome during cell division¹³; this is also demonstrated by maintenance of plasmid containing the KSHV latent origin of replication (ori-P) in transfected cells¹⁴. In addition to histone H2A/B¹⁵, LANA interacts with many other chromatin-binding proteins; those include bromodomain-containing proteins 2 and 4 (BRD2/4)¹⁶, KDM3A¹⁷, hSET1 complex¹⁸, MLL1 complex¹⁹, and CHD4²⁰. These interactions regulate histone occupancies and active/suppressive histone modifications at LANA complex recruited sites. LANA oligomerization, which is likely to be facilitated by DNA binding at TR with increased protein concentration, was found to be essential for many of the protein-protein interaction²¹. Among LANA interacting proteins, CHD4 knock-down displayed one of the strongest phenotypes in KSHV gene expression²⁰. The studies showed that the knock-down of CHD4 enhances KSHV reactivation and inhibits entering latency in de nova infected 293 cells²⁰. Consistent with the observation, recent CHD4 studies for the host chromatin binding sites also showed that CHD4 primarily localizes at epigenetically active chromatins and suppresses activation of cellular enhancer and promoter activity by preventing the formation of active genomic hubs with enhancers^{22, 23}.

Enhancers are distal DNA regulatory elements that regulate promoter activity through a looping mechanism by which enhancers are brought into proximity to their promoters^{24, 25}. This mechanism supports the frequent interaction of enhancer-bounded mediators with transcription factors on promoters in the events of activation at either promoter or enhancer elements²⁶. The enhancers often display a high density of transcription factor binding sites to have the flexibility to respond to external stimuli²⁷, and their regulators such as co-activator enzymes and/or suppressors^{20, 28-30}. CHD4 is one of the enzymes that regulate enhancer accessibility²³, which is found in two distinct cellular repressor complexes, the NuRD and ChAHP, and the ChAHP complex plays essential roles in maintaining accurate cell fate decisions during development by regulating enhancer accessibility³¹⁻³³. Genetic disruption of CHD4 therefore causes spontaneous differentiation concomitant with premature activation of lineage-specific genes³²⁻³⁴.

Dysregulation of the gene transcription program by mutations and/or overexpression of transcription-related enzymes prevents cell differentiation and frequently leads to cell transformation³⁵⁻³⁷. Consistent with this, overexpression of CHD4 is associated with poor progression in hepatocellular carcinoma³⁸, colorectal cancer³⁹, and ovarian cancer⁴⁰. CHD4 is also identified as essential for breast cancer cell growth, and CHD4 depletion induces a G0/G1 block of the cell cycle with up-regulation of CDKN1A (p21)⁴¹. In all cases, depletion of CHD4 decreases cancer cell proliferation, in some cases, through induction of autophagic cell death⁴². Taken together, inhibition of CHD4, which facilitates cell differentiation by reactivation of enhancers, would be an approach to target the dedifferentiated cancer cells.

Viruses have made their way into human bodies, constantly adapting to their host cell environment to live together. The lineage of viruses dates back to 450 million years⁴³, and their uniqueness lies in the fact that they have to utilize host cell enzymes to produce progenies. Viral proteins, therefore, often possess a higher affinity protein interaction domain to outcompete host cell proteins to transcribe or replicate their genomes within a short time with limited protein coding capability^{44, 45}. Identification of such specific protein domains may therefore be used as a competitive inhibitor of interacting host proteins^{44, 46}.

In this study, we isolated a biologically active peptide from the binding interface between LANA and CHD4. The introduction of the small peptide induces caspase-dependent cleavage of CHD4, facilitates cell differentiation in the monocyte differentiation model, and prevents cancer cell growth in vitro and xenograft PEL mouse model. The comprehensive characterization from identification of peptide sequence to xenograft studies is described in this report.

### Results

Identification of CHD4 Interacting Peptide from KSHV LANA Protein Sequence

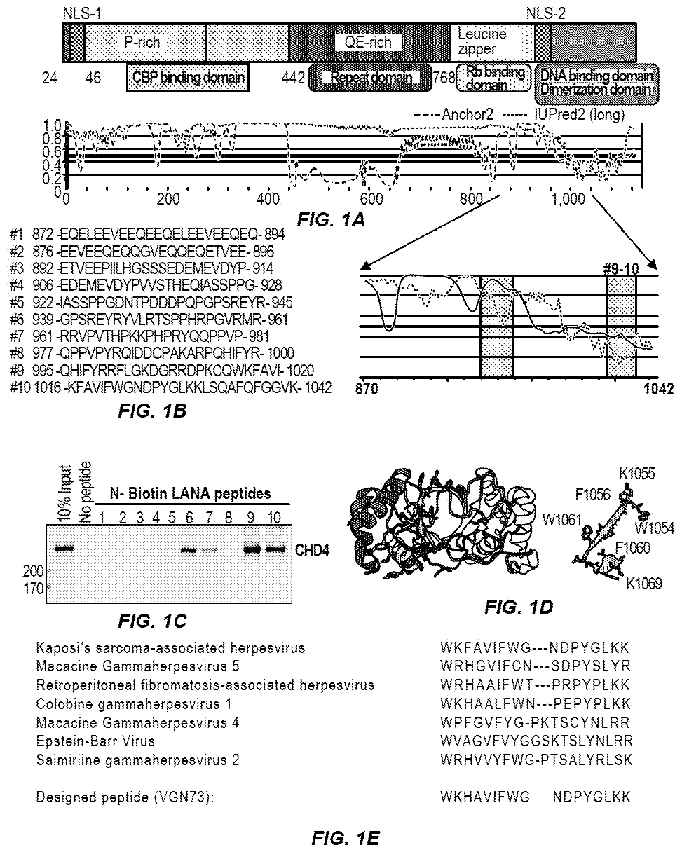

We previously identified KSHV LANA interacting protein with proximity biotin labeling approaches by generating recombinant KSHV²⁰. The proteomics study identified that CHD4 interacts with LANA and co-occupied on both host and viral chromatins²⁰. Biochemical mapping studies further showed that the amino acid sequence between 870 and 1042 of LANA was responsible for the interaction (FIG. 1a). The interaction domain was located relatively structured domains, even though LANA contains very large intrinsically disordered domain at acidic repeat region (FIG. 1a). To narrow the interaction surface with CHD4, a series of overlapped N-terminal biotinylated peptides were prepared (FIG. 1b), and peptide pull-down studies were performed. The results showed that amino acid sequence between 945 and 961 and between 1,000 and 1042 was able to precipitate purified Flag-CHD4 protein (FIG. 1c).

Based on the previously published LANA C-terminal crystal structure^{47, 48}, we visualized the position of the peptide sequence responsible for the CHD4 interaction. The results showed that the CHD4 interaction surface is located between DNA binding interface and the LANA homo dimerization domain (FIG. 1d), indicating that CHD4 may be recruited very close to chromatins. If the function of the CHD4 interaction is important, we speculate that there will be a consensus amino acid sequence for the interaction that are evolutionally maintained with other gamma-herpesviruses. The homologous proteins from other gamma herpesviruses were therefore aligned with KSHV LANA. The alignment indeed showed highly conserved tryptophan, tyrosine, and leucine, which is next to two charged amino acids (arginine or lysine) (FIG. 1e). The C-terminal basic amino acids are predicted to contact DNA^{47, 48}. Highly hydrophobic or aromatic residues were also conserved among gamma herpesvirus functional homologs. Based on consensus motifs, we synthesized a small peptide, which also encodes cell-penetrating peptide (TAT) to introduce the small peptide into cells. We designed a peptide by an amino acid change into LANA protein sequence, which makes it less hydrophobic for better cell penetrating efficacies. To increase the half-life of the peptide, we also included D-amino acids at N-terminus, which has been shown to prevent rapid degradation by host cell enzymes⁴⁹. The final design of the cell-penetrating LANA peptide is shown in FIG. 1e. We later found that the peptide has cancer cell-killing activity, we named the peptide VGN73 (Virus de Gann wo Naosu ORF73), which means, in Japanese, utilizing virus's wisdom to combat cancers.

### Characterization of LANA Peptide

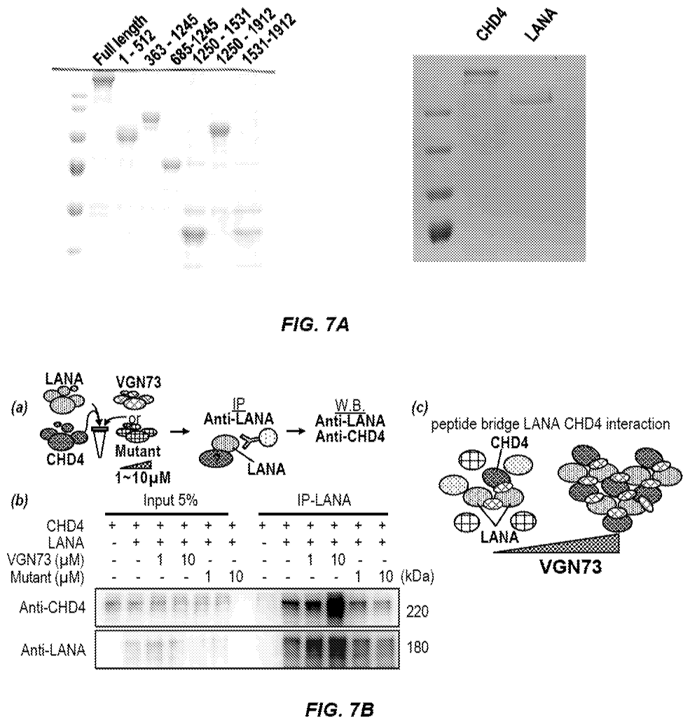

We next examined peptide binding sites on the CHD4 domain and measured affinity to further confirm the interaction. Multiple CHD4 deletion proteins were expressed and purified from recombinant baculovirus-infected Sf9 cells with Flag-agarose beads (FIG. 7a). An equal molecular amount of recombinant proteins was coated on an ELISA plate and increasing concentrations of biotinylated peptides were incubated. The biotinylated peptide bound to recombinant CHD4 that encodes 1-512, 363-1245, or the fill-length CHD4, while the peptide failed to bind recombinant proteins lacking PHD domain (FIG. 2a). The results indicate that the peptide may bind to CHD4 PHD domain, which is known to interact with acetylated H3 histone tail⁵⁰. Binding affinity to the fill-length CHD4 was also calculated with Bio-Layer Interferometry (BLI). To demonstrate the specificity of the interaction, we also generated a mutant peptide, which contains three amino acid changes at conserved amino acids (FIG. 2b). The N-biotinylated VGN73 or control peptides were first immobilized on a streptavidin-coated chip, and sequentially diluted full-length Flag-CHD4 proteins were applied. The interaction was then detected with interferometry. The results showed that VGN73 but not mutant or TAT alone binds to CHD4, and the affinity of the interaction was calculated to be approximately 13.5 nM. The results also indicate that the interaction is sequence-specific, because mutations eliminated the interaction (FIG. 2c).

The position of VGN73 locates next to the LANA homo dimerization domain, we next asked if the presence of the LANA peptide could influence the CHD4-LANA interactions. The mutant peptide or no peptide incubated was used as a negative control. Two purified proteins (FIG. 7b) were first incubated in the presence or absence of peptides, and the protein complex was precipitated with an anti-LANA antibody, and interaction was probed with immunoblotting. The results showed that presence of VGN73. CHD4 interacted more with LANA, and a larger amount of LANA was also precipitated in presence of peptide, indicating that the peptide facilitates the interactions among LANA and CHD4 in vitro (FIG. 7c). Next the observation was examined in KSHV naturally-infected primary effusion lymphoma (PEL) cells. First, efficacies of cell penetration were confirmed by Cy5 labeled VGN73 or its mutant. Both peptides effectively penetrated cells within 60 min, and there are no noticeable differences in peptide distribution in cells (FIG. 2d). To evaluate if the peptide binds to CHD4 or LANA in tissue culture, we next incubated with biotinylated peptides for 1 hour, and peptide and peptide interacting proteins were precipitated with streptavidin magnetic beads. The amount of peptide-bound CHD4 or LANA was then probed with a specific antibody. The VGN73 precipitated more of CHD4 and LANA than the overall background, although the amount of precipitated CHD4 or LANA was relatively small in the experiment condition (FIG. 2e). Taken together, these results showed that the small peptide isolated from LANA-CHD4 interaction was able to bind CHD4 in vitro and in cells.

### VGN73 Induces Caspase-Mediated CHD4 Cleavage and Decreases the Amount of CHD4 and LANA in PELs

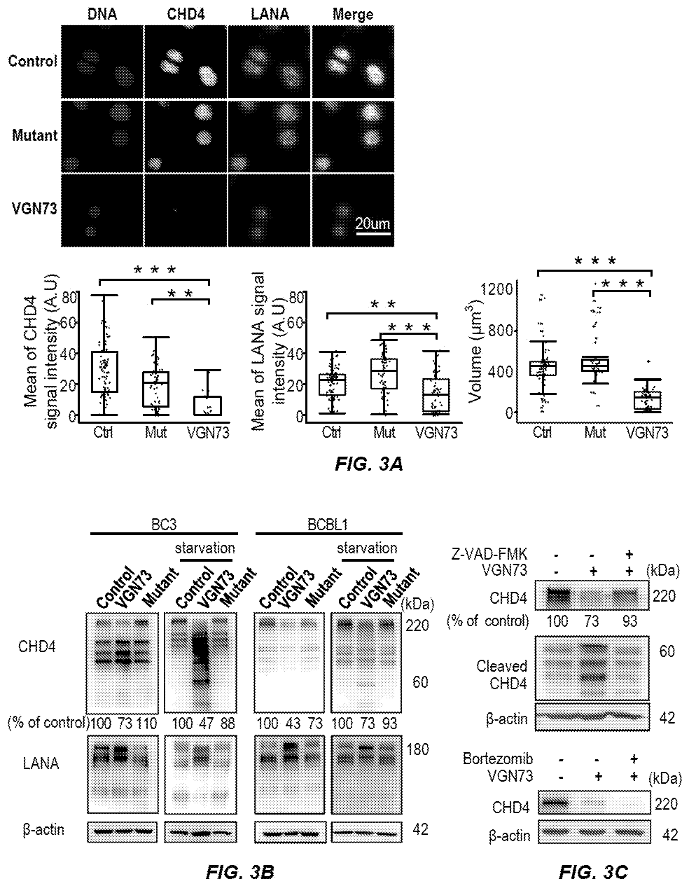

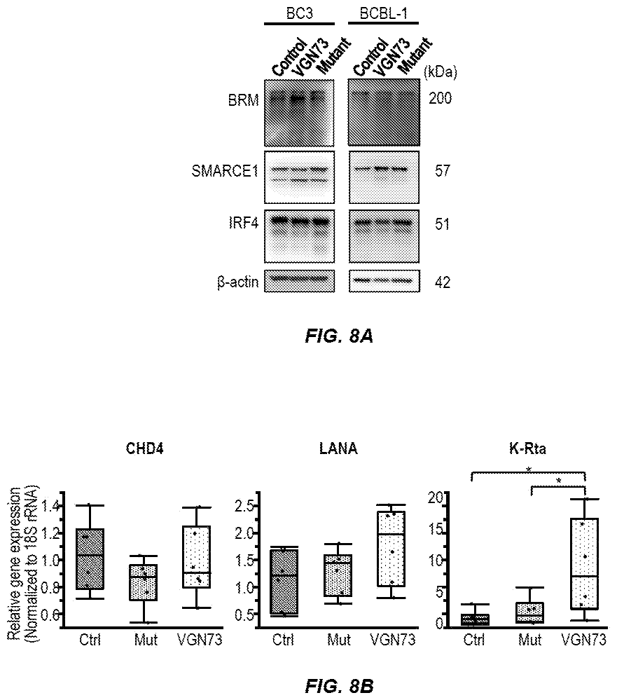

We next examined the biological effects of the VGN73 on CHD4 and LANA in BC3 and BCBL-1 cells. The peptides were incubated with IC₅₀ concentration. The CHD4 and LANA were then stained with specific antibodies. The results showed that the intensity of the CHD4 signal was substantially lowered in BC3 cells treated with VGN73 but not with mutant (FIG. 3a), which is correlated with a decreased amount of full length of CHD4 in total cell lysates (FIG. 3b). LANA also showed different migration patterns in VGN73-treated cells. While presumable full-length CHD4 was decreased, the intensity of smaller forms of CHD4 was increased, indicating induction of CHD4 cleavages in presence of VGN73 (FIG. 3b). The protein degradation was not seen with other SWI/SNF family proteins, BRM, SMARCE1, or a transcription factor IRF4 in total lysates (FIG. 8a). A pretreatment with Z-VAD-FMK, a pan-caspase inhibitor, but not a proteasome inhibitor, recovered full-length CHD4 (FIG. 3c). The results show that CHD4 is cleaved by caspase in presence of VGN73. Interestingly, under a low serum condition, CHD4 cleavage was enhanced (FIG. 3b). The results indicatee that induction of cell stress further increases VGN73-mediated CHD4 cleavage, which indicates the involvement of autophagy.

### Lana Peptide Induces Cell Apoptosis and Autophagy in Leukemia Cells

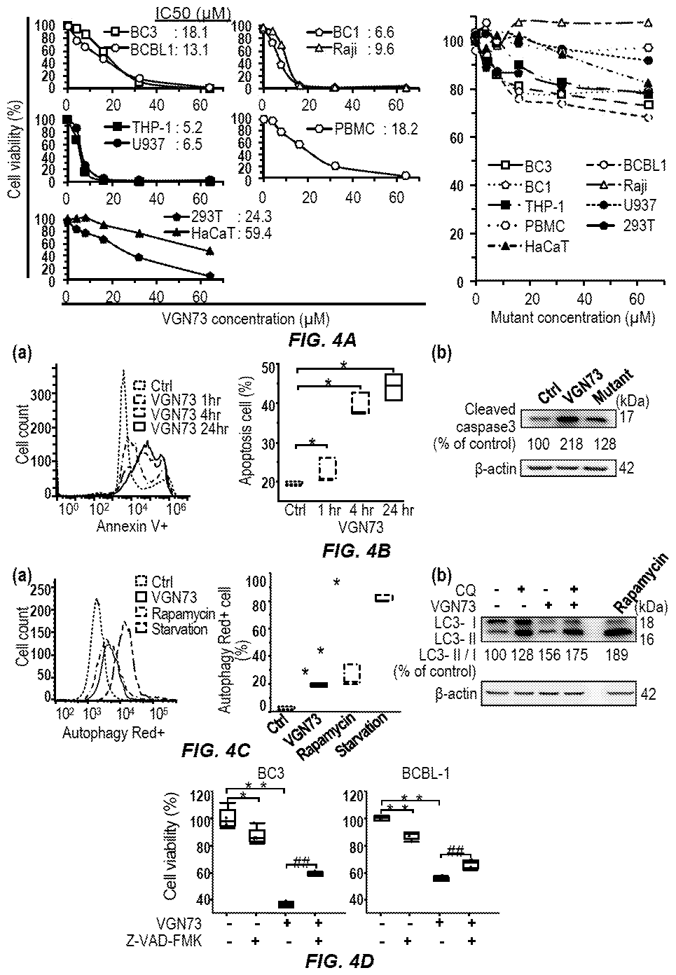

CHD4 is overexpressed in multiple cancer types and, in some cases, CHD4 was suggested to be a driver for tumorigenesis²². Cleavage of CHD4 protein with LANA peptide prompted us to examine general therapeutic effects with cancer cell lines. Because the VGN73 is isolated from the KSHV protein sequence, we first tested if VGN73 inhibits primary effusion lymphoma (PEL) growth. PEL is a KSHV-associated B-cell lymphoma⁵¹. An increasing amount of VGN73 was incubated, and the cell viability was indirectly measured by MTT assays. The results showed that VGN73 inhibited PEL cell growth in a dose-dependent manner (FIG. 4a). Interestingly, other leukemic cancer cell lines are even more sensitive to VGN73, suggesting that the effects of cell killing are independent of KSHV infection. PEL cells that are infected with KSHV only, are less sensitive to the VGN73, compared with cell lines harboring the EBV genome (Raji, BC1). Two monocyte leukemia cells, THP-1 and U937 cells had the highest sensitivity to the VGN73. Interestingly, HaCaT cells, immortalized keratinocytes, are resistant to VGN73, and normal PBMCs are also relatively resistant compared with monocytic leukemia cell lines. Flow cytometry analyses with Annexin V or Autophagy Red showed that VGN73 induces cellular apoptosis and also auto-phagosome formation (FIG. 4b, c). The VGN73 treatment induced autophagy in BC3 cells similarly to rapamycin treatment, which was used as a positive control (FIG. 4c). Western blotting also indicated increasing of apoptosis and autophagy markers. Importantly, pretreating with Z-VAD-FMK partially rescued BC3 and BCBL-1 from cell death, even though Z-VAD-FMK treatment alone reduced cell viability for more than 10% (FIG. 4d). These results demonstrated while exact molecular mechanism of VGN73-mediated cancer cell killing remained elusive, VGN73 but not mutant peptide effectively induces cancer cell death in vitro tissue culture by triggering cellular apoptosis and autophagy, and these two pathways are perhaps mutually exclusive⁵².

Identification of Direct Targets of VGN73 with SLAM-Seq

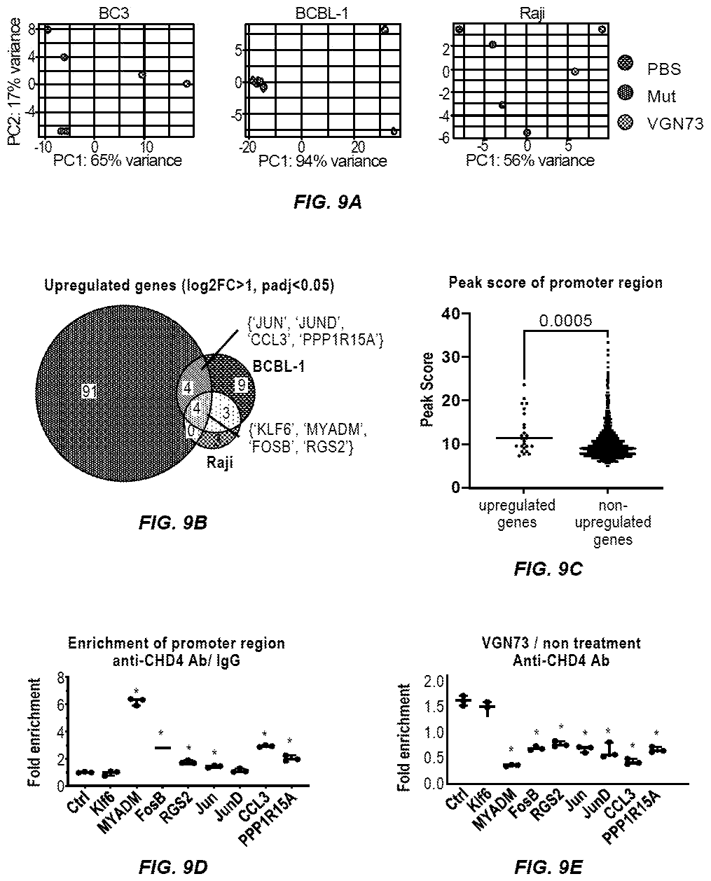

To better understand the biological activity and molecular actions of VGN73, we next employed two platforms for transcription analyses. The SLAM-seq with three B-cell lines was first employed to isolate direct targets from secondary effects through indirect activation or repression⁵³. For direct target gene identification, we used mutant peptide and vehicle-treated cells as comparisons, and, by studying three cell lines, commonly up-regulated gene sets were identified. The results showed that KLF6, MYADM, FOSB, RGS2, JUN, JUND, CCL3, and PPP1R15A are commonly upregulated among three cell lines (FIG. 9b). To reveal association with CHD4 occupancies, we next analyzed the CHD4 peak score at promoter regions of the upregulated genes that are found as direct targets of VGN73 with SLAM-seq. The results showed that promoters of upregulated gene were significantly more enriched with CHD4 than those of non-upregulated gene (FIG. 9c). Furthermore, we also examined the occupancies of CHD4 on each promoter by CUT&RUN-qPCR in BCBL-1 cells. As the negative control, non-CHD4 bound region and control IgG were used. The promoter regions of VGN73-target genes showed enrichment of CHD4 (FIG. 9d), and the VGN73 incubation indeed decreased the occupancies of CHD4 from each promoter, except for Klf6 (FIG. 9e). These results indicate VGN73 treatment primarily targets CHD4, which unleashes poised cellular immediate-early gene expression. The Gene Set Enrichment Analyses of the direct-target genes showed that enrichment of skeletal muscle cell differentiation related genes that regulate cell shapes in Raji and BCBL-1 cells, while BC3 cells did not have statistically significant specific pathway enrichment.

### VGN73 Facilitates Monocyte Cell Differentiation to Macrophages

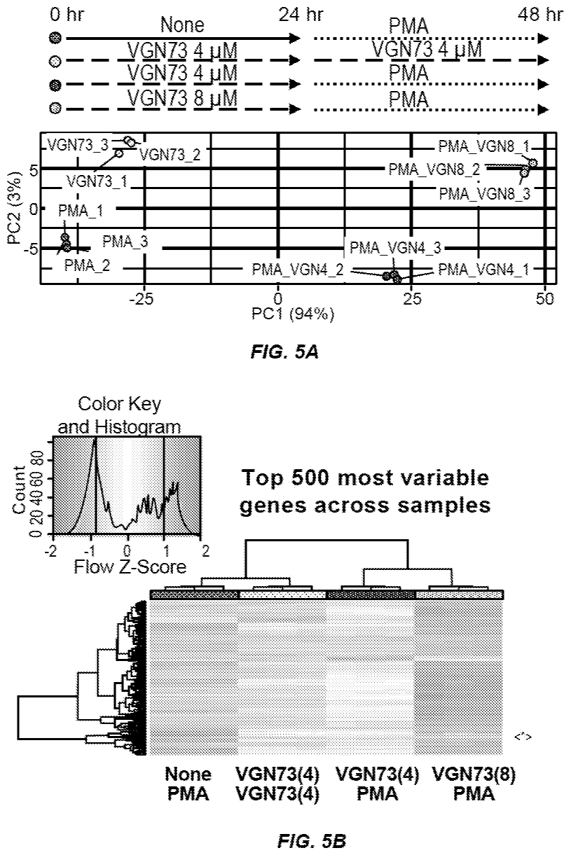

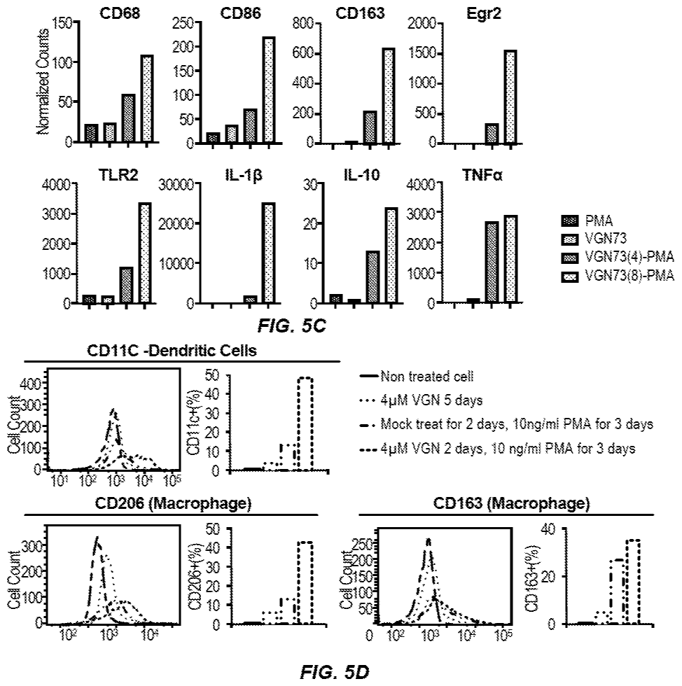

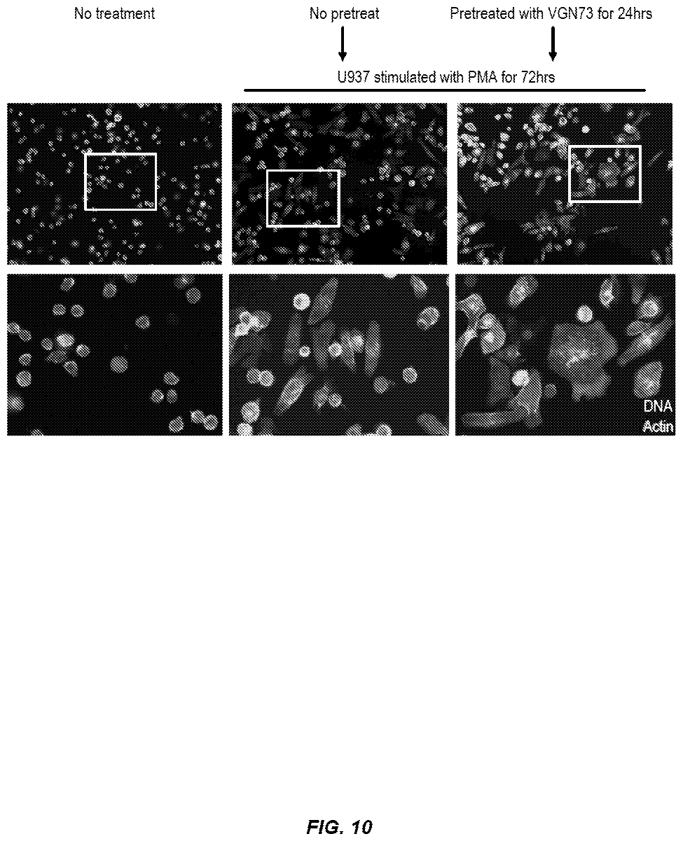

The CHD4 is known to regulate cell lineage by restricting cell differentiation^{33, 54}. Accordingly, we next specifically examined the effects of VGN73 in regulation of cell differentiation with U937 monocyte-macrophage model⁵⁵. The U937 cell model was selected, because (i) U937 cells were one of the most sensitive cell lines to the VGN73 incubation in MT assays, and (ii) we frequently saw that U937 displayed the characteristics of macrophage differentiation such as increased cell adhesion, and large cytoplasm during experiments (FIG. 10). The FIG. 5a depicted our experiment scheme, the phorbol-12-myristate-13-acetate (PMA), an analog of diacyl-glycerol, was used for a macrophage differentiation inducer^{55, 56}, and we monitored if VGN73 pretreatment can further facilitate by reducing CHD4 occupancies from gene regulatory regions. The U937 cells were pre-treated with 4 or 8 μM VGN73 for 24 hours, and PMA was added thereafter for another 24 hours. The VGN73 or PMA treatment alone was used as a comparison. While VGN73 or PMA alone displayed similar gene expression patterns except highly selective gene sets (FIG. 5b with asterisk). However, pretreating with VGN73 significantly increased the repertories of PMA-mediated upregulated genes, and those effects were appeared not to be additive. A closer look at VGN73 pre-treated samples showed substantially increased overall activated transcripts that include genes involved in macrophage differentiation and inflammatory cytokines, when we compared with PMA without VGN73-pretreat (FIG. 5b, c). The flow cytometry independently confirmed increased macrophage and dendritic cell marker on the cell surface of VGN73-pretreated U937 cells (FIG. 5d). These transcriptomics and genomics studies suggested that VGN73 treatment reduces occupancies of CHD4 on promoter, which increased accessibility of transcriptional factors to upregulate gene expression.

### VGN73 Prevents PEL Cell Growth in the Xenograft Model and Reduces CHD4 and LANA Expression

Induction of cell differentiation in immature cancer cells is one of the therapeutic strategies, called differentiation therapy, to reduce tumorigenesis⁵⁷. Terminal differentiation of cancer stem cells or the conversion into non-stem cells increases the sensitivity of tumors to conventional anticancer treatments and also prevents metastasis⁵⁸. Perhaps due to the function of CHD4 in restricting cell differentiation. CHD4 is frequently found overexpressed in many cancer types^{40, 59}. We thus examined the utility of VGN73 as a therapeutic drug in a xenograft mouse model.

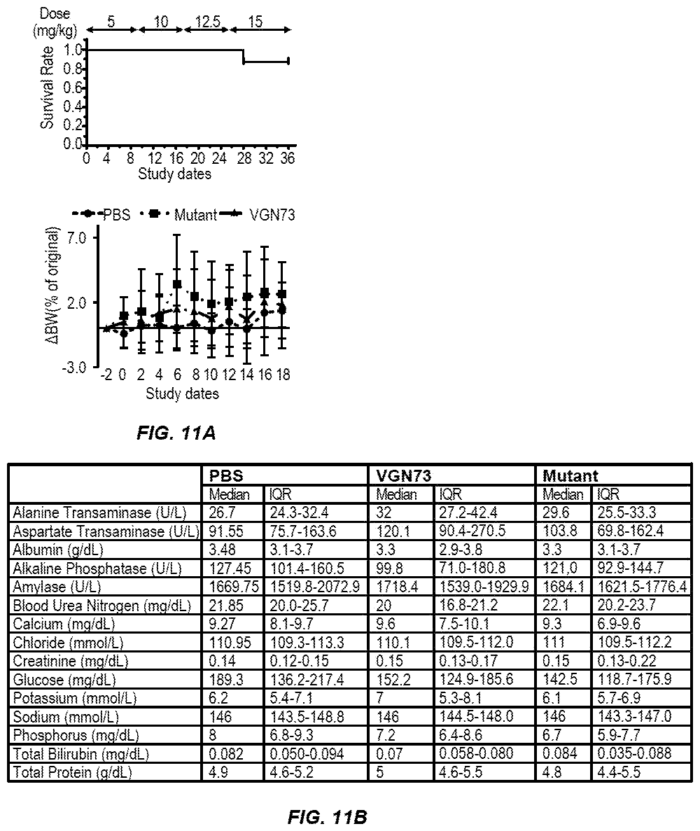

We first examined the maximum tolerated dose in mice by injecting an increasing amount of VGN73 intraperitoneally. The results found that injections of VGN73 of more than 15 mg/kg showed a sign of discomfort such as decreased overall mobility, and one of eight mice died the next day (FIG. 11e). Accordingly, we considered 12.5 mg/kg as the maximum tolerated dose and decided to use 10 mg/kg every other day as a treatment schedule. With the treatment schedule, there was no decreased body weight over three weeks, and blood chemistry showed no sign of organ damage (FIG. 11b).

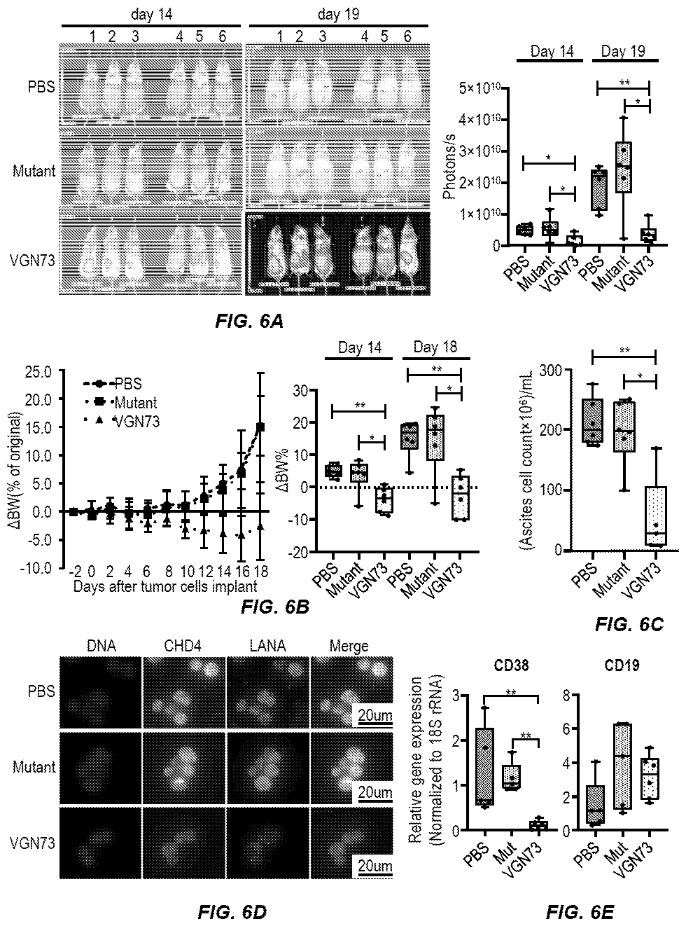

With a PEL mouse xenograft model, we inoculated 5×10⁶ luciferase-expressing BCBL-1 cells intraperitoneally. Two days after the PEL inoculation, we started to treat with VGN73, mutant peptide, or vehicle (PBS) in IP (10 mg/kg/every other day). PEL cell growth was monitored with bioluminescence imaging on days 14 and 19. The results showed that in the VGN73-treated group. BCBL-1 growth was substantially inhibited. One of the mutant peptide-treated mice did not show significant luciferase activity and we later found that BCBL-1 was primarily grown subcutaneously. Increasing body weight by BCBL-1 produced fluid, which is an indication of BCBL-1 cell growth, was also consistent with the imaging analyses (FIGS. 6a and 6b). At Day 19, ascites fluid was collected and the total number of PEL cells in the fluid was counted. Because very little ascites fluid was produced in VGN73 treated group, we recovered cells by applying PBS and collected residual cells. The results directly confirmed the inhibition of PEL growth in xenograft mice (FIG. 6c). Collected PEL cells from xenograft tumors were then stained with CHD4 and LANA. The results showed that VGN73 treatment lowered both CHD4 and LANA signals in PEL cells in mice, which is consistent with in vitro studies (FIGS. 3a & 6d). Finally, gene expressions of cell surface marker were examined for recovered PEL cells from ascites. The results showed that CD38, an immature marker and indicating potent proliferating capability, was significantly downregulated in VGN73 treated cells (FIG. 6e). On the other hand, CD19, a selected B cell differentiation marker, was slightly increased, although it showed significant variations within treated mouse and did not meet statistical significance (FIG. 6e). Taken together. VGN73 is a biologically active peptide, which degrades CHD4 and remodels the cellular gene expression program. With a therapeutic dose, VGN73 prevents PEL growth, perhaps by facilitating terminal cell differentiation in immature cancer cells, with no measurable side effects in mice.

### DISCUSSION

This study utilizes the fact that viruses depend upon host cell machineries for producing infectious progenies. Accordingly, viral proteins have acquired the ability to control many cellular functions under continuous pressure from host defense mechanisms during millions of years of co-evolutions⁶⁰. Therefore, it is not surprising to find a functional small peptide from the viral protein sequence, which has strong biological activity with high affinity to the host cell proteins.

The identified VGN73 is a 17-amino acid peptide, which is derived from the KSHV LANA sequence. During the screening, we noticed that C-terminal 6 amino acids (PYGLKK) were not essential for the leukemic cell-killing activity, but the addition of the segment increases the cell-killing effects approximately 2-fold. Accordingly, in this study, we use the longer version of the VGN73. In addition, while an original KSHV LANA protein sequence has phenylalanine (F) at the third position, changing to less hydrophobic histidine, which is conserved in other gamma-herpesvirus homologs, increased peptide solubility in water and found it better cell membrane penetration. Additional modifications such as the inclusion of non-natural amino acids at the C-terminus for increasing peptide stability would likely to increase the biological activity of the VGN73.

VGN73 bound to purified CHD4 with ˜14 nM K_{D}, which is a relatively high affinity considering the size of the peptide. Tissue culture studies also showed that VGN73 effectively induces caspase-mediated CHD4 cleavages, while it spares other cellular proteins such as actin and other nuclear proteins. BRM. SMARCE1 and IRF4. We could not rule out a contribution of inducing cell stress responses by the VGN73, which would also explain caspase activation; however, mutant peptides that have three amino acids change, which equally penetrated cultured cells, did not have such effects. Specific and narrowly targeted gene activation with VGN73 with SLAM-seq also supports specificities. In addition, these highly upregulated target gene promoters were indeed occupied by CHD4, and VGN73 treatment reduced its occupancies. Based on these results, we propose VGN73's biological function is, in part, through inhibition of CHD4. Further large-scale proteomics studies would detail the molecular actions of VGN73.

Among selected cancer cell types, we found monocytic cell lines (U937, THP-1) were the most sensitive to the VGN73, and PEL cell lines that express the KSHV LANA protein were relatively resistant. We also noticed that the presence of EBV infection seems to sensitize VGN73 treatment. HaCaT cell and PBMC, those relatively normal cells were not very sensitive to VGN73. We speculate that cells that maintain undifferentiated status with CHD4 by restricting promoter activation may determine the sensitivity to VGN73. Cells that are already differentiated (e.g., keratinocyte, peripheral blood mononuclear cell) might be relatively unaffected by the treatment. This may also explain very little side effects to mice. Consistent with this, it has been reported that CD34 positive AML is highly sensitive to CHD4 depletion⁶¹.

The direct target gene identification by SLAM-seq indicates that treating VGN73 preferentially releases the immediate-early transcription factor expression. These immediate-early genes are poised to be expressed in response to a variety of external stimuli, and VGN73 treatment appears to release promoters from such transcription repression. This is similar to what we have seen in KSHV latent chromosomes with CHD4 knock-down m, and VGN73 incubation indeed increased K-Rta-mediated lytic gene expression (FIG. 8b). Specific to the BC3 cells, we also see the induction of TP53 gene expression; this partially explains the activation of number of cellular apoptosis related gene expression cell apoptosis, and having different transcription profile (FIG. 9b). BC3 is known to encode wild-type TP53, while BCBL-1 cells and Raji cells have mutations in TP53^{62, 63}.

To our surprise, pre-incubation with VGN73 drastically changed a list of activated genes with PMA in the top 500 most variable genes. We speculate that such large changes may attribute to activation of dormant enhancers that are restricted by CHD4 complex. Further studies with HiChIP (HiC and chromatin immunoprecipitation)⁶⁴ should clarify the effects on enhancer-promoter interactions with VGN73.

U937 cell differentiation model by RNA-seq indicated that VGN73 enhanced differentiation from monocytes to macrophages and/or dendric cells. Moreover, in xenografted mice model, an immature marker which relate to cell proliferation was significantly decreased on BCBL-1 cells recovered from ascites of mice treated with VGN73. Cancer cell plasticity is known to drive cancer progression and cancer sternness to reversely convert their identity associated with drug resistance⁶³. The concept of cell differentiation therapy is from the fact that terminal cell differentiation irreversibly changes the phenotype and makes the cancer cell sensitive to conventional chemotherapeutic drugs. The differentiation therapy is based on the great success in acute promyelocytic leukemia (APL)⁶⁶. The APL became highly curable with the combination of retinoic acid and arsenic treatment, which stimulates oncoprotein degradation to induce terminal differentiation of leukemic cells into granulocytes⁶⁷. Similarly, many new therapies for myeloid malignanciesenter clinical practice, including epigenetic therapies (e.g., 5-azacitidine), isocitrate dehydrogenase inhibitors, fms-like kinase 3 inhibitors, and lenalidomide for deletion 5q (del5q) myelodysplastic syndrome⁶⁸ were found to induce, and the cell differentiation is considered to be a major mechanism by which several of these functions as cancer therapeutics. CHD4 is a critical factor to restrict cell differentiation and known to maintain embryonic stem cell identity by controlling differentiation-associated genes^{33, 33, 54, 69}. In our study, MYADM, whose promoter region was enriched for CHD4 occupancy and were the most affected by VGN73 treatment, is a known as marker of myeloid differentiation⁷⁰. In addition, considering cell differentiation render cancer cells to be sensitive to conventional chemotherapy⁴⁰, VGN73 is likely to synergize with some cancer drugs.

In summary, our study demonstrates that viral proteins are a unique starting material as the basis for designing therapeutics directed at attenuating cellular protein function(s). The VGN73 peptides are useful in targeting specific cell types as antibody-conjugates or may use as a tool to enhance cell differentiation of inducible pluripotent stem cells in a combination with specific stimuli.

### Methods and Materials

### Cell Culture

BC1, BC3, BCBL-1, Raji, THP-1, U937, 293T, and HaCaT cell lines were obtained from ATCC. Leukoreduction system chambers (LRS) from healthy donors were purchased from Vitalant. Peripheral blood mononuclear cells (PBMCs) were prepared by a standard Ficoll gradient method. These cell lines were cultured in RPMI 1640 medium supplemented with 15% FBS, antibiotics, and L-glutamine, or IMDM medium supplemented with 15% FBS, antibiotics, and L-glutamine for BC-3 cell lines, or DMEM medium supplemented with 10% FBS, antibiotics, and L-glutamine for 293T and HaCaT cell lines.

### Enzyme Linked Immunosorbent Assays (ELISA)

To evaluate the VGN73 binding region of CHD4, ELISA was carried out. Biotin-conjugated VGN73 was diluted with phosphate buffered saline (PBS) (pH=7.0) to a final concentration of 1 μM. Each well of a 96-well flat-bottom streptavidin-coated microplate (bioWORLD, Dublin, OH, USA) was coated with 100 μL of biotin-conjugated VGN73 in PBS overnight at 4° C. Each region of flag conjugated-CHD4 was tested at the following concentrations: 3.1, 6.3, 12.5, 25, 50, 100 nM, and vehicle only (0 nM). The wells were washed three times with Tris-buffered saline containing 0.1% Tween-20 (TBS-T) with 0.1% bovine serum albumin (BSA). Blocking buffer (5% BSA in TBS-T) was added, and the plate was incubated at 37° C. for 1 h. After washing the wells three times as described above, 200 μL of different concentrations of each region of flag conjugated-CHD4 diluted with 0.1% BSA in TBS-T were applied to each well. The plate was incubated at room temperature for 2 h and washed 5 times with TBS-T. Bound proteins were probed with 200 μL of streptavidin-horseradish peroxidase (HRP) conjugate (ThermoFisher Scientific, Waltham, MA. USA), which was diluted 1:20,000 in 0.1% BSA in TBS-T and applied to each well. After incubation at room temperature for 1.5 h and washing 5 times with TBS-T, color development with the TMB Substrate (ThermoFisher Scientific, Waltham, MA. USA) was performed according to the manufacture's protocol. The optical densities (ODs) were measured at 450 nm with a Benchmark Plus Microplate Spectrophotometer (Bio-Rad, Hercules. CA. USA). The assays were performed in triplicate wells. Each absorbance was calculated by subtracting absorbance of the blank from the measured value in each experimental well.

### Biolayer Interferometry (BLI)

To evaluate of binding kinetics of VGN73 and mutant peptide to CHD4, assays were carried out by biolayer interferometry (BLI) on Octet-Red 384 (ForteBio, CA, USA) at 30° C. with shaking at 1,000 RPM. Streptavidin (SA) tips (Sartorius, Goettingen, Germany) were dipped in 200 μL of biotinylated peptide (VGN73, mutant or TAT) solution (1 μM in 1× kinetic buffer) for the loading step. The tips loaded with peptide were then sampled with CHD4 at various concentrations in 1× kinetic buffer (Sartorius, Goettingen, Germany) to obtain the association curve. TAT was used as a reference for background subtraction. After association, the tips were dipped back into 1× kinetic buffer to obtain the dissociation curve. Binding kinetics was evaluated using a 1:1 binding model by ForteBio Data Analysis 8.1 software to obtain the dissociation constant K_{D}.

### Peptide-Pulldown Assay

One assay with anti-LANA antibody was performed to evaluate that VGN73 binds between CHD4 and LANA in vitro. Flag-LANA and Flag-CHD4 were prepared by recombinant baculoviruses, and each protein was isolated in the presence of 500 mM NaCl and 2% glycerol with affinity purification (FIG. 7b). LANA (200 nM) and CHD4 (200 nM) were incubated with VGN73 (1 or 10 μM) or mutant peptide (1 or 10 μM) in 200 μL binding buffer (20 mM HEPES [pH 7.9]. 150 mM NaCl, 1 mM EDTA, 4 mM MgCl₂, 1 mM dithiothreitol, 0.02% NP-40, 10% glycerol supplemented with 1 mg/1 mL BSA, and 13 protease inhibitor cocktail) and the anti-LANA antibody was incubated at 1:100 dilution for 1 h at 4° C. to form an immunocomplex. The immunocomplex was captured with 10 μL of protein G magnetic beads. Another assay with streptavidin beads was performed to evaluate that VGN73 binds CHD4 and LANA in tissue culture. BC3 cells were incubated with biotinylated VGN73 (10 μM), biotinylated mutant peptide (10 μM) or biotinylated TAT peptide (10 μM) for 4 hours. Cells were washed with cold PBS 3 times followed by lysed with RIPA lysis buffer (150 mM NaCl, 5 mM EDTA (pH 8). 50 mM Tris (pH 8). 1% NP-40, 0.5% sodium deoxycholate. 0.1% SDS). Lysates were immediately boiled for 10 min, and protein was quantified by Bradford assay (Bio-Rad). Affinity purification was performed with streptavidin-coated magnetic beads (Thermo Fisher). Briefly, 50 μL magnetic beads/sample were pre-washed with Tris-buffered saline containing 0.1% Tween-20 (TBS-T) 3 times. A total of 1.5 mg of whole cell lysate was incubated with pre-washed streptavidin beads, at room temperature for 1 hour and 4° C. overnight with rotation. The beads were collected using a magnetic stand and washed three times with TBS-T according to the manufacturer's protocol. Finally, in each assay, beads were washed 3 times and eluted antigen from beads in 40 μL of SDS-PAGE sample buffer by boiling for 5 min. The respective protein band was confirmed by immunoblotting with anti-CHD4 and anti-LANA antibodies.

In Vitro Cell Treatment with VGN73

BC3 cells and BCBL-1 cells were treated with VGN73 at the concentration of IC50 for 24 hours. These cells were also treated with VGN73 in media without FBS. To evaluate the effect of VGN73 on CHD4 and LANA proteins in tissue culture, IFA and western blotting were carried out, and these gene expressions were evaluated by RT-qPCR. Moreover, BC3 cells were pretreated with pan-caspase inhibitor, Z-VAD-FMK (50 μM) or proteasome inhibitor, bortezomib (25 nM) for 1 hour, followed by treated with VGN73 (18 μM) for 8 hours. BC3 cells were pretreated with lysosome inhibitor, chloroquine (7.5 μM) for 1 hour, followed by treated with VGN73 (18 μM) for 24 hours. Western blotting was carried out for analyzing biological mechanisms for CHD4 decreasing and inducing cell death by VGN73 treatment.

U937 cells were pretreated with 0, 4 or 8 μM of VGN73 for 24 hours, followed by stimulated with PMA (10 ng/ml) for another 24 hours. To evaluate a molecular action of VGN73, total RNA-sequencing analysis with monocyte differentiation model with U937 cell line was carried out. Moreover, U937 cells were pretreated with 4 μM of VGN73 for 2 days, followed by a stimulate with 10 ng/ml of PMA for another 3 days. Cell treated with VGN73 for 2 days or, PMA for 3 days as controls. The cell surface markers for differentiation were evaluated by Flow cytometry.

### Immunofluorescence Staining Analyses (IFA) and Imaging Analyses

To evaluate subcellular peptide localization, IFA was carried out (FIG. 2d). Cy5-labeled peptides were used to track subcellular localization. BC3 cells were treated with 10 μM of Cy5-labeled VGN73 or mutant peptide for 1 min and 60 min. Moreover, to evaluate VGN73 efficacy to CHD4 and LANA in tissue culture. IFA was carried out (FIG. 3a). BC3 cells were treated with VGN73 at a concentration of IC50 or mutant peptide for 24 hours. In both assays, the cells were washed with PBS 2 times after peptide treatment. The cells were then fixed with 4% formaldehyde, permeabilized with 0.2% Triton X-100. In the assay evaluating VGN73 efficacy, labeled with anti-CHD4 rabbit monoclonal antibody (Cell Signaling, D4B7; 1:100) and anti-LANA rat monoclonal antibody (Millipore-Sigma, clone LN53: 1:200), followed by Alexa 555-anti rat IgG and Alexa 647-anti rabbit IgG (Thermo Fisher). Nuclei were counterstained with 1 μg/ml of Hoechst 33342 (Thermo Fisher). The labeled cells were observed with a Keyence BZ-X710 fluorescence microscope (Keyence, Osaka, Japan) with standard DAPI, GFP, TRITC, and Cy5 filter sets (Chroma Technologies, Bellows Falls, VT, USA). The exposure settings and image acquisition protocols were fixed in each imaging study to enable quantitative analysis. Mean LANA and CHD4 fluorescence (measured in arbitrary units, or A.U.) per cell was subsequently calculated using onboard software and/or NIH Image (FIG. 3a(b)).

### Western Blotting

Cells were washed twice with PBS and lysed with the protein lysis buffer (50 mM Tris-HCl [pH 6.8], 2% SDS, 10% glycerol). The lysates were boiled in SDS-PAGE loading buffer and subjected to SDS-PAGE, and subsequently transferred to a polyvinylidene fluoride membrane (Millipore-Sigma, St. Louis, MO. USA) using a semidry transfer apparatus (Bio-Rad. Hercules. CA. USA). The streptavidin-HRP conjugate was used at 1:3000 dilution. Final dilutions of the primary antibodies were 1:2.000 for anti-β-actin mouse antibody, 1:1,000 for anti-LANA rat antibody, anti-CHD4 rabbit, anti-cleaved caspase 3, rabbit anti-LC3 rabbit, anti-BMR rabbit, anti-SMARCE1 rabbit, and anti-IRF4 rabbit antibodies. Membrane washes and secondary antibody incubations were performed according to methods described in the literature.

### RT-qPCR

BC3 cells treated with VGN73 and BCBL-1 cells from ascites of xenografted mice were used for RT-qPCR. Cells were washed with PBS two times. Total RNA was isolated using the Quick-RNA miniprep kit (Zymo Research. Irvine. CA, USA). First-strand cDNA was synthesized using the High Capacity cDNA Reverse Transcription Kit (Thermo Fisher. Waltham, MA USA). Gene expression was analyzed by real-time qPCR using specific primers: CHD4. LANA and K-Rta for BC3 cells, and CD19 and CD38 for BCBL-1 cells. We used 18S ribosomal RNA as an internal standard to normalize viral gene expression.

### Cell Viability Assay

The cytotoxic activity of VGN73 and mutant peptide were measured using the MTT assay. BC1, BC3, BCBL1, Raji, THP-1. U937, 293T, HaCaT cells, and PBMC from a healthy donor were treated with VGN73 or mutant peptide at various concentrations (0, 4, 8, 16, 32, 64 μM) for 24 hours. These cells were incubated with MTT (0.5 mg/mL) at 37° C. for 4 hours, and lysed with 10% SDS containing lysis buffer for overnight. The optical densities (ODs) were measured at 570 nm with a Benchmark Plus Microplate Spectrophotometer (Bio-Rad). For caspase inhibition study. BC3 and BCBL-1 cells were pretreated with 40 μM of Z-VAD-FMK; pan caspase inhibitor for 1 hour before incubating with VGN73 for 4 hours.

### Flow Cytometry

BC3 cells were treated with 18 μM of VGN73 for 0, 1, 4, and 24 hours. Apoptosis was evaluated after intracellular staining with annexin-V-FITC and 7-AAD (Biolegend) according to the manufacture's protocol. Apoptosis (%) was determined as AnnexinV+ 7AAD-population. Autophagy induction by treating with 18 μM of VGN73 for 24 hours was evaluated with intracellular staining with autophagy probe red (Bio-Rad, Hercules, CA, USA) according to the manufacture's protocol. BC3 cells were also treated with 50 nM of rapamycin or cells were cultured under serum starved condition as positive controls. U937 cells were treated with 4 μM of VGN73 for 5 day, 10 ng/ml of PMA for 3 days, or pretreated with 4 μM of VGN73 for 2 days, then stimulated with 10 ng/ml of PMA for 3 days. Cell differentiations to dendritic cell and macrophage were evaluated after staining with CD11C, CD206 or CD163. Flow cytometry was carried out by using a BD Acuri instrument (BD Biosciences) and the data was analyzed with FlowJo v10.8.0 (Tree Star) software.

### RNA-Sequencing

Indexed, stranded mRNA-seq libraries were prepared from total RNA (100 ng) using the KAPA Stranded mRNA-Seq kit (Roche) according to the manufacturer's standard protocol. Libraries were pooled and multiplex sequenced on an Illumina NovaSeq 6000 System (150-bp, paired-end, >30×106 reads per sample). RNA-Seq data was analyzed using a Salmon-tximport-DESeq2 pipeline. Raw sequence reads (FASTQ format) were mapped to the reference human genome assembly (GRCh38/hg38. GENCODE release 36) and quantified with Salmon. Gene-level counts were imported with tximport and differential expression analysis was performed by DEseq2, which are visualized by volcano plot.

### Cleavage Under Targets and Release Using Nuclease (CUT&RUN)

CUT&RUN⁷¹ was performed essentially by following the online protocol developed by Dr. Henikoff's lab with a few modifications to fit our needs. Cells were washed with PBS and wash buffer [20 mM HEPES-KOH pH 7.5, 150 mM NaCl, 0.5 mM Spermidine (Sigma, S2626), and proteinase inhibitor (Roche)]. After removing the wash buffer, cells were captured on magnetic concanavalin A (ConA) beads (Polysciences, PA, USA) in the presence of CaCl2. Beads/cells complexes were washed three times with digitonin wash buffer (0.02% digitonin, 20 mM HEPES-KOH pH 7.5, 150 mM NaCl. 0.5 mM Spermidine and 1× proteinase inhibitor), aliquoted, and incubated with anti-CHD4 antibody or human IgG in 250 μL volume. The antibodies and concentrations used in this study was: rabbit monoclonal anti-CHD4 (Cell Signaling, D4B7: 1:50). After incubation, unbound antibody was removed by washing with digitonin wash buffer three times. Beads were then incubated with recombinant Protein A/G-Micrococcal Nuclease (pAG-MNase), which was purified from E. coli in 250 μl digitonin wash buffer at 1.0 μg/mL final concentration for 1 h at 4° C. with rotation. Unbound pAG-MNase was removed by washing with digitonin wash buffer three times. Pre-chilled digitonin wash buffer containing 2 mM CaCl₂ (200 μL) was added to the beads and incubated on ice for 30 min. The pAG-MNase digestion was halted by the addition of 200 μl 2× STOP solution (340 mM NaCl, 20 mM EDTA, 4 mM EGTA, 50 μg/ml RNase A, 50 μg/ml glycogen). The beads were incubated with shaking at 37° C. for 10 min in a tube shaker at 500 rpm to release digested DNA fragments from the insoluble nuclear chromatin. The supernatant was collected after centrifugation (16,000×g for 5 min at 4° C.) and placed on a magnetic stand. DNA was extracted using the NucleoSpin Gel & PCR kit (Takara Bio, Kusatsu, Shiga, Japan). Sequencing libraries were then prepared from 3 μg of CUT&RUN DNA with the Kapa HyperPrep Kit (Roche) according to the manufacturer's standard protocol. Libraries were multiplex sequenced (2-150 bp, paired-end) on an Illumina HiSeq 4000 sequencing system to yield ˜15 million mapped reads per sample. The extracted DNA also was used to examine enrichment at selected genomic regions by qPCR (CUT&RUN-qPCR). All PCR data was normalized by 10% of the input reaction before CUT& RUN. As the negative control, non-CHD4 bound region and control IgG were used. CUT&RUN-qPCR data were analyzed by using the 2^{-ΔΔCT} method and compared with NC.

### SLAM-Seq

SLAM-seq was performed using the SLAMseq Kinetics Kit (Lexogen GmbH, Vienna. Austria) according to the manufacturer's standard protocol. Briefly, biological replicate cultures of BC3, BCBL-1 or Raji cells were incubated with VGN73 peptide (at each concentration of IC50) for 30 min. Subsequently, 4-Thiouridine (s4U; 300 μM) was added to the culture media and the cells incubated for 1.5 hours in order to label newly synthesized RNA. Total RNA was isolated and then the 4-thiol groups in the s4Uracil-labeled transcripts were alkylated with iodoacetamide (IAA). QuantSeq 3′ mRNA-Seq (FWD) (Lexogen. Inc.) Illumina-compatible, indexed sequencing libraries were prepared from alkylated RNA samples (100 ng) according to the manufacturer's protocol for oligo(dT)-primed first strand cDNA synthesis, random-primed second strand synthesis, and library amplification. Libraries were multiplex sequenced (1×100 bp, single read) on an Illumina HiSeq 4000 sequencing system. SLAM-Seq datasets were analyzed using the T>C conversion-aware SLAMDUNK (Digital Unmasking of Nucleotide conversion-containing k-mers) pipeline utilizing the default parameters. Briefly, nucleotide conversion-aware read mapping of adapter- and poly(A)-trimmed sequences to the human GRCh38/hg38 reference genome assembly was performed with NextGenMap. Alignments were filtered for those with a minimum identity of 95% and minimum of 50% of the read bases mapped. For multi-mappers, ambiguous reads and non-3′ UTR alignments were discarded, while one read was randomly selected from multimappers aligned to the same 3′ UTR. SNP calling (coverage cut-off of 10× and variant fraction cutoff of 0.8) with VarScan2 was performed in order to mask actual T>C SNPs. Non-SNP T>C conversion events were then counted and the fraction of labeled transcripts determined. All results were exported (i.e., tcount file) and used for downstream analyses, such as principal component analysis and differential expression analysis (DESeq2). For visualization, RefSeq IDs were converted to official gene symbols (refGene). The resulting data were first filtered by Log 2FC>=1 and sorted by Padj from lowest to highest (<0.01). The resulting differentially-expressed genes in BC3, BCBL-1 and Raji were illustrated in a Venn diagram.

### PEL Xenografts

All animal studies were conducted according to a UC Davis Institutional Animal Care and Use Committee (IACUC)-approved protocol. NRG (NOD.Cg-Rag1tm1Mom Il2rgtm1Wjl/SzJ) mouse breeding pairs were purchased from the Jackson Laboratory and the colony was maintained in-house. To evaluate the in vivo antitumor activity of VGN73, twenty-three to thirty-week-old female NRG mice were injected intraperitoneally (i.p.) with 5×10⁶ BCBL-1 luciferase cells in 500 μL PBS. On day 2, mice were randomly assigned to PBS control, VGN73 (10 mg/kg), or mutant control peptide (10 mg/kg) groups. Treatments were administered by every other day i.p. injection for 18 days. Mice were monitored for PEL burden based on body weight increment. At day 14 and 19 post-xenografted, mice were intraperitoneally injected with D-luciferin at 2 mg and imaged with Lago X. Images were analyzed using Aura ver 4.0.7. Mice were sacrificed and recovered ascites for analysis of ascites cell count, CHD4 and LANA protein expression by IFA and gene expression of CD38 and CD19 by RT-qPCR.

### Visualization of KSHV LANA C-Terminal Domain

The dimer of LANA C-terminal domain (PDB: 2YPY)⁴⁷, the structure determined by the X-ray crystal structure, were visualized and highlighted with the molecular visualization open-source software program PyMOL (Ver 2.5.0).

### Statistics and Reproducibility

Results are shown as mean±SD or median [interquartile range (IQR)], from at least three independent experiments. Data were analyzed using Wilcoxon signed-rank test. A value of p<0.05 was defined as statistically significant. Statistical analyses were conducted using JMP version 13.2.1. For animal experiments, sample size is based on our previous study⁴⁴.

### REFERENCES

[1] Chang, Y., Cesarman, E., Pessin, M. S., Lee, F., Culpepper, J., Knowles, D. M., and Moore, P. S. (1994) Identification of herpesvirus-like DNA sequences in AIDS-associated Kaposi's sarcoma, Science 266, 1865-1869.
[2] Schalling, M., Ekman, M., Kaaya, E. E., Linde, A., and Biberfeld, P. (1995) A role for a new herpes virus (KSHV) in different forms of Kaposi's sarcoma, Nat Med 1, 707-708.
[3] Cesarman, E., Chang, Y., Moore, P. S., Said, J. W., and Knowles, D. M. (1995) Kaposi's sarcoma-associated herpesvirus-like DNA sequences in AIDS-related body-cavity-based lymphomas, N Engl J Med 332, 1186-1191.
[4] Nador, R. G., Cesarman, E., Chadburn, A., Dawson, D. B., Ansari, M. Q., Said, J., and Knowles, D. M. (1996) Primary effusion lymphoma: a distinct clinicopathologic entity associated with the Kaposi's sarcoma-associated herpes virus, Blood 88, 645-656.
[5] Soulier, J., Grollet, L., Oksenhendler, E., Cacoub, P., Cazals-Hatem, D., Babinet, P., d'Agay, M. F., Clauvel, J. P., Raphael, M., Degos, L., and et al. (1995) Kaposi's sarcoma-associated herpesvirus-like DNA sequences in multicentric Castleman's disease, Blood 86, 1276-1280.
[6] Oksenhendler, E., Carcelain, G., Aoki, Y., Boulanger, E., Maillard, A., Clauvel, J. P., and Agbalika, F. (2000) High levels of human herpesvirus 8 viral load, human interleukin-6, interleukin-10, and C reactive protein correlate with exacerbation of multicentric castleman disease in HIV-infected patients, Blood 96, 2069-2073.
[7] Uldrick, T. S., Wang, V., O'Mahony, D., Aleman, K., Wyvill, K. M., Marshall, V., Steinberg, S. M., Pittaluga, S., Maric, I., Whitby, D., Tosato, G., Little, R. F., and Yarchoan, R. (2010) An interleukin-6-related systemic inflammatory syndrome in patients co-infected with Kaposi sarcoma-associated herpesvirus and HIV but without Multicentric Castleman disease, Clin Infect Dis 51, 350-358.
[8] Polizzotto, M. N., Uldrick, T. S., Wang, V., Aleman, K., Wyvill, K. M., Marshall, V., Pittaluga, S., O'Mahony, D., Whitby, D., Tosato, G., Steinberg, S. M., Little, R. F., and Yarchoan, R. (2013) Human and viral interleukin-6 and other cytokines in Kaposi sarcoma herpesvirus-associated multicentric Castleman disease, Blood 122, 4189-4198.
[9] Yan, L., Majerciak, V., Zheng, Z. M., and Lan, K. (2019) Towards Better Understanding of KSHV Life Cycle: from Transcription and Posttranscriptional Regulations to Pathogenesis, Virol Sin 34, 135-161.
[10] Iftode, N., Radulescu, M. A., Arama, S. S., and Arama, V. (2020) Update on Kaposi sarcoma-associated herpesvirus (KSHV or HHV8)—review, Rom J Intern Med 58, 199-208.
[11] Sandhu, P. K., and Damania, B. (2022) The regulation of KSHV lytic reactivation by viral and cellular factors, Curr Opin Virol 52, 39-47.
[12] Uppal, T., Banerjee, S., Sun, Z., Verma, S. C., and Robertson, E. S. (2014) KSHV LANA—the master regulator of KSHV latency, Viruses 6, 4961-4998.
[13] Barbera, A. J., Chodaparambil, J. V., Kelley-Clarke, B., Luger, K., and Kaye, K. M. (2006) Kaposi's sarcoma-associated herpesvirus LANA hitches a ride on the chromosome, Cell Cycle 5, 1048-1052.
[14] Shinohara, H., Fukushi, M., Higuchi, M., Oie, M., Hoshi, O., Ushiki, T., Hayashi, J., and Fujii, M. (2002) Chromosome binding site of latency-associated nuclear antigen of Kaposi's sarcoma-associated herpesvirus is essential for persistent episome maintenance and is functionally replaced by histone H1, J Virol 76, 12917-12924.
[15] Barbera, A. J., Chodaparambil, J. V., Kelley-Clarke, B., Joukov, V., Walter, J. C., Luger, K., and Kaye, K. M. (2006) The nucleosomal surface as a docking station for Kaposi's sarcoma herpesvirus LANA, Science 311, 856-861.
[16] Ottinger, M., Christalla, T., Nathan, K., Brinkmann, M. M., Viejo-Borbolla, A., and Schulz, T. F. (2006) Kaposi's sarcoma-associated herpesvirus LANA-1 interacts with the short variant of BRD4 and releases cells from a BRD4- and BRD2/RING3-induced G1 cell cycle arrest, J Virol 80, 10772-10786.
[17] Kim, K. Y., Huerta, S. B., Izumiya, C., Wang, D. H., Martinez, A., Shevchenko, B., Kung, H. J., Campbell, M., and Izumiya, Y. (2013) Kaposi's sarcoma-associated herpesvirus (KSHV) latency-associated nuclear antigen regulates the KSHV epigenome by association with the histone demethylase KDM3A, J Virol 87, 6782-6793.
[18] Hu, J., Yang, Y., Turner, P. C., Jain, V., McIntyre, L M., and Renne, R. (2014) LANA binds to multiple active viral and cellular promoters and associates with the H3K4methyltransferase hSET1 complex, PloS Pathog 10, e1004240.
[19] Tan, M., Li, S., Juillard, F., Chitas, R., Custodio, T. F., Xue, H., Szymula, A., Sun, Q., Liu, B., Alvarez, A. L., Chen, S., Huang, J., Simas, J. P., McVey, C. E., and Kaye, K. M. (2021) MLL1 is regulated by KSHV LANA and is important for virus latency, Nucleic Acids Res 49, 12895-12911.
[20] Kumar, A., Lyu, Y., Yanagihashi, Y., Chantarasrivong, C., Majerciak, V., Salemi, M., Wang, K. H., Inagaki, T., Chuang, F., Davis, R. R., Tepper, C. G., Nakano, K., Izumiya, C., Shimoda, M., Nakajima, K. I., Merleev, A., Zheng, Z. M., Campbell, M., and Izumiya, Y. (2022) KSHV episome tethering sites on host chromosomes and regulation of latency-lytic switch by CHD4, Cell Rep 39, 110788.
[21] De Leo, A., Deng, Z., Vladimirova, O., Chen, H. S., Dheekollu, J., Calderon, A., Myers, K. A., Hayden, J., Keeney, F., Kaufer, B. B., Yuan, Y., Robertson, E., and Lieberman, P. M. (2019) LANA oligomeric architecture is essential for KSHV nuclear body formation and viral genome maintenance during latency, PLoS Pathog 15, e1007489.
[22] Marques, J. G., Gryder, B. E., Pavlovic, B., Chung, Y., Ngo, Q. A., Frommelt, F., Gstaiger, M., Song, Y., Benischke, K., Laubscher, D., Wachtel, M., Khan, J., and Schafer, B. W. (2020) NuRD subunit CHD4 regulates super-enhancer accessibility in rhabdomyosarcoma and represents a general tumor dependency, Elife 9.
[23] Goodman, J. V., Yamada, T., Yang, Y., Kong, L., Wu, D. Y., Zhao, G., Gabel, H. W., and Bonni, A. (2020) The chromatin remodeling enzyme Chd4 regulates genome architecture in the mouse brain, Nat Commun 11, 3419.
[24] Dorsett, D. (1999) Distant liaisons: long-range enhancer-promoter interactions in Drosophila, Curr Opin Genet Dev 9, 505-514.
[25] Dorsett, D., and Merkenschlager, M. (2013) Cohesin at active genes: a unifying theme for cohesin and gene expression from model organisms to humans, Curr Opin Cell Biol 25, 327-333.
[26] Panigrahi, A., and O'Malley, B. W. (2021) Mechanisms of enhancer action: the known and the unknown, Genome Biol 22, 108.
[27] Saini, A., Ghoneim, H. E., Lio, C. J., Collins, P. L, and Oltz, E. M. (2022) Gene Regulatory Circuits in Innate and Adaptive Immune Cells, Annu Rev Immunol 40, 387-411.
[28] Gasperini, M., Tome, J. M., and Shendure, J. (2020) Towards a comprehensive catalogue of validated and target-linked human enhancers, Nat Rev Genet 21, 292-310.
[29] Langer, L. F., Ward, J. M., and Archer, T. K. (2019) Tumor suppressor SMARCB1 suppresses super-enhancers to govern hESC lineage determination, Elife 8.
[30] Kubo, N., Ishii, H., Xiong, X., Bianco, S., Meitinger, F., Hu, R., Hocker, J. D., Conte, M., Gorkin, D., Yu, M., Li, B., Dixon, J. R., Hu, M., Nicodemi, M., Zhao, H., and Ren, B. (2021) Promoter-proximal CTCF binding promotes distal enhancer-dependent gene activation, Nat Struct Mol Biol 28, 152-161.
[31] Zhao, H., Han, Z., Liu, X., Gu, J., Tang, F., Wei, G., and Jin, Y. (2017) The chromatin remodeler Chd4 maintains embryonic stem cell identity by controlling pluripotency- and differentiation-associated genes, J Bio Chem 292, 8507-8519.
[32] Ostapcuk, V., Mohn, F., Carl, S. H., Basters, A., Hess, D., Iesmantavicius, V., Lampersberger, L., Flemr, M., Pandey, A., Thoma, N. H., Betschinger, J., and Buhler, M. (2018) Activity-dependent neuroprotective protein recruits HP1 and CHD4 to control lineage-specifying genes, Nature 557, 739-743.
[33] Arends, T., Dege, C., Bortnick, A., Danhorn, T., Knapp, J. R., Jia, H., Harmacek, L., Fleenor, C. J., Straign, D., Walton, K., Leach, S. M., Feeney, A. J., Murre, C., O'Connor, B. P., and Hagman, J. R. (2019) CHD4 is essential for transcriptional repression and lineage progression in B lymphopoiesis, Proc Natl Acad Sci USA 116, 10927-10936.
[34] Sun, X., Yu, W., Li, L, and Sun, Y. (2020) ADNP Controls Gene Expression Through Local Chromatin Architecture by Association With BRG1 and CHD4, Front Cell Dev Biol 8, 553.
[35] Lidschreiber, K., Jung, L. A., von der Emde, H., Dave, K., Taipale, J., Cramer, P., and Lidschreiber, M. (2021) Transcriptionally active enhancers in human cancer cells, Mol Syst Biol 17, e9873.
[36] Li, G. H., Qu, Q., Qi, T. T., Teng, X. Q., Zhu, H. H., Wang, J. J., Lu, Q., and Qu, J. (2021) Super-enhancers: a new frontier for epigenetic modifiers in cancer chemoresistance, J Exp Clin Cancer Res 40, 174.
[37] Mirzadeh Azad, F., and Atlasi, Y. (2021) Deregulation of Transcriptional Enhancers in Cancer, Cancers (Bosel) 13.
[38] Nio, K., Yamashita, T., Okada, H., Kondo, M., Hayashi, T., Hara, Y., Nomura, Y., Zeng, S. S., Yoshida, M., Hayashi, T., Sunagozaka, H., Oishi, N., Honda, M., and Kaneko, S. (2015) Defeating EpCAM(+) liver cancer stem cells by targeting chromatin remodeling enzyme CHD4 in human hepatocellular carcinoma, J Hepatol 63, 1164-1172.
[39] Xia, L, Huang, W., Bellani, M., Seidman, M. M., Wu, K., Fan, D., Nie, Y., Cai, Y., Zhang, Y. W., Yu, L. R., Li, H., Zahnow, C. A., Xie, W., Chiu Yen, R. W., Rassool, F. V., and Baylin, S. B. (2017) CHD4 Has Oncogenic Functions in Initiating and Maintaining Epigenetic Suppression of Multiple Tumor Suppressor Genes, Cancer Cell 31, 653-668 e657.
[40] Oyama, Y., Shigeta, S., Tokunaga, H., Tsuji, K., Ishibashi, M., Shibuya, Y., Shimada, M., Yasuda, J., and Yaegashi, N. (2021) CHD4 regulates platinum sensitivity through MDR1 expression in ovarian cancer: A potential role of CHD4 inhibition as a combination therapy with platinum agents, PLoS One 16, e0251079.
[41] D'Alesio, C., Punzi, S., Cicalese, A., Fornasari, L, Furia, L, Riva, L, Carugo, A., Curigliano, G., Criscitiello, C., Pruneri, G., Pelicci, P. G., Faretta, M., Bossi, D., and Lanfrancone, L (2016) RNAi screens identify CHD4 as an essential gene in breast cancer growth, Oncotarget 7, 80901-80915.
[42] D'Alesio, C., Bellese, G., Gagliani, M. C., Lechiara, A., Dameri, M., Grasselli, E., Lanfrancone, L, Cortese, K., and Castagnola, P. (2019) The chromodomain helicase CHD4 regulates ERBB2 signaling pathway and autophagy in ERBB2(+) breast cancer cells, Biol Open 8.
[43] Aiewsakun, P., and Katzourakis, A. (2017) Marine origin of retroviruses in the early Palaeozoic Era, Nat Commun 8, 13954.
[44] Shimoda, M., Lyu, Y., Wang, K. H., Kumar, A., Miura, H., Meckler, J. F., Davis, R. R., Chantarasrivong, C., Izumiya, C., Tepper, C. G., Nakajima, K. I., Tuscano, J., Barisone, G., and Izumiya, Y. (2021) KSHV transactivator-derived small peptide traps coactivators to attenuate MYC and inhibits leukemia and lymphoma cell growth, Commun Biol 4, 1330.
[45] Chen, C. P., Lyu, Y., Chuang, F., Nakano, K., Izumiya, C., in, D., Campbell, M., and Izumiya, Y. (2017) Kaposi's Sarcoma-Associated Herpesvirus Hijacks RNA Polymerase II To Create a Viral Transcriptional Factory, J Virol 91.
[46] Lee, J. S., Li, Q., Lee, J. Y., Lee, S. H., Jeong, J. H., Lee, H. R., Chang, H., Zhou, F. C., Gao, S. J., Liang, C., and Jung, J. U. (2009) FLIP-mediated autophagy regulation in cell death control, Nat Cell Biol 11, 1355-1362.
[47] Hellert, J., Weidner-Glunde, M., Krausze, J., Richter, U., Adler, H., Fedorov, R., Pietrek, M., Ruckert, J., Ritter, C., Schulz, T. F., and Luhrs, T. (2013) A structural basis for BRD2/4-mediated host chromatin interaction and oligomer assembly of Kaposi sarcoma-associated herpesvirus and murine gammaherpesvirus LANA proteins, PLoS Pathog 9, e1003640.
[48] Domsic, J. F., Chen, H. S., Lu, F., Marmorstein, R., and Lieberman, P. M. (2013) Molecular basis for oligomeric-DNA binding and episome maintenance by KSHV LANA, PLoS Pathog 9, e1003672.
[49] Hong, S. Y., Oh, J. E., and Lee, K. H. (1999) Effect of D-amino acid substitution on the stability, the secondary structure, and the activity of membrane-active peptide, Biochem Pharmacol 58, 1775-1780.
[50] Jain, K., Fraser, C. S., Marunde, M. R., Parker, M. M., Sagum, C., Burg, J. M., Hall, N., Popova, I. K., Rodriguez, K. L, Vaidya, A., Krajewski, K., Keogh, M. C., Bedford, M. T., and Strahl, B. D. (2020) Characterization of the plant homeodomain (PHD) reader family for their histone tail interactions, Epigenetics Chromatin 13, 3.
[51] Said, W., Chien, K., Takeuchi, S., Tasaka, T., Asou, H., Cho, S. K., de Vos, S., Cesarman, E., Knowles, D. M., and Koeffler, H. P. (1996) Kaposi's sarcoma-associated herpesvirus (KSHV or HHV8) in primary effusion lymphoma: ultrastructural demonstration of herpesvirus in lymphoma cells, Blood 87, 4937-4943.
[52] Thorburn, A. (2008) Apoptosis and autophagy: regulatory connections between two supposedly different processes, Apoptosis 13, 1-9.
[53] Herzog, V. A., Reichholf, B., Neumann, T., Rescheneder, P., Bhat, P., Burkard, T. R., Wlotzka, W., von Haeseler, A., Zuber, J., and Ameres, S. L (2017) Thiol-linked alkylation of RNA to assess expression dynamics, Nat Methods 14, 1198-1204.
[54] Sreenivasan, K., Rodriguez-delaRosa, A., Kim, J., Mesquita, D., Segales, J., Arco, P. G., Espejo, I., Ianni, A., Di Croce, L, Relaix, F., Redondo, J. M., Braun, T., Serrano, A. L, Perdiguero, E., and Munoz-Canoves, P. (2021) CHD4 ensures stem cell lineage fidelity during skeletal muscle regeneration, Stem Cell Reports 16, 2089-2098.
[55] Garcia, A., Serrano, A., Abril, E., Jimenez, P., Real, L M., Canton, J., Garrido, F., and Ruiz-Cabello, F. (1999) Differential effect on U937 cell differentiation by targeting transcriptional factors implicated in tissue- or stage-specific induced integrin expression, Exp Hematol 27, 353-364.
[56] Cavender, D. E., Edelbaum, D., and Welkovich, L. (1991) Effects of inflammatory cytokines and phorbol esters on the adhesion of U937 cells, a human monocyte-like cell line, to endothelial cell monolayers and extracellular matrix proteins, J Leukoc Biol 49, 566-578.
[57] Arima, Y., Nobusue, H., and Saya, H. (2020) Targeting of cancer stem cells by differentiation therapy, Cancer Sci 111, 2689-2695.
[58] Shiozawa, Y., Nie, B., Pienta, K. J., Morgan, T. M., and Taichman, R. S. (2013) Cancer stem cells and their role in metastasis, Pharmacol Ther 138, 285-293.
[59] Wang, H. C., Chou, C. L., Yang, C. C., Huang, W. L., Hsu, Y. C., Luo, C. W., Chen, T. J., Li, C. F., and Pan, M. R. (2019) Over-Expression of CHD4 Is an Independent Biomarker of Poor Prognosis in Patients with Rectal Cancers Receiving Concurrent Chemoradiotherapy, Int J Mol Sci 20.
[60] Sharp, P. M., and Simmonds, P. (2011) Evaluating the evidence for virus/host co-evolution, Curr Opin Virol 1, 436-441.
[61] Heshmati, Y., Turkoz, G., Harisankar, A., Kharazi, S., Bostrom, J., Dolatabadi, E. K., Krstic, A., Chang, D., Mansson, R., Altun, M., Qian, H., and Walfridsson, J. (2018) The chromatin-remodeling factor CHD4 is required for maintenance of childhood acute myeloid leukemia, Haematologica 103, 1169-1181.
[62] Roy, D., Sin, S. H., Damania, B., and Dittmer, D. P. (2011) Tumor suppressor genes FHIT and WWOX are deleted in primary effusion lymphoma (PEL) cell lines, Blood 118, e32-39.
[63] Duthu, A., Debuire, B., Romano, J., Ehrhart, J. C., Fiscella, M., May, E., Appella, E., and May, P. (1992) p53 mutations in Raji cells: characterization and localization relative to other Burkitt's lymphomas, Oncogene 7, 2161-2167.
[64] Mumbach, M. R., Rubin, A. J., Flynn, R. A., Dai, C., Khavari, P. A., Greenleaf, W. J., and Chang, H. Y. (2016) HiChIP: efficient and sensitive analysis of protein-directed genome architecture, Nat Methods 13, 919-922.
[65] Miranda, A., Hamilton, P. T., Zhang, A. W., Pattnaik, S., Becht, E., Mezheyeuski, A., Bruun, J., Micke, P., de Reynies, A., and Nelson, B. H. (2019) Cancer stemness, intratumoral heterogeneity, and immune response across cancers, Proc Natl Acad Sci USA 116, 9020-9029.
[66] de The, H. (2018) Differentiation therapy revisited, Nat Rev Cancer 18, 117-127.
[67] de The, H., Pandolfi, P. P., and Chen, Z. (2017) Acute Promyelocytic Leukemia: A Paradigm for Oncoprotein-Targeted Cure, Cancer Cell 32, 552-560.
[68] Stubbins, R. J., and Karsan, A. (2021) Differentiation therapy for myeloid malignancies: beyond cytotoxicity, Blood Cancer 11, 193.
[69] Hirota, A., Nakajima-Koyama, M., Ashida, Y., and Nishida, E. (2019) The nucleosome remodeling and deacetylase complex protein CHD4 regulates neural differentiation of mouse embryonic stem cells by down-regulating p53, J Biol Chem 294, 195-209.
[70] Wang, Q., U, N., Wang, X., Shen, J., Hong, X., Yu, H., Zhang, Y., Wan, T., Zhang, L, Wang, J., and Cao, X. (2007) Membrane protein hMYADM preferentially expressed in myeloid cells is up-regulated during differentiation of stem cells and myeloid leukemia cells, Life Sci 80, 420-429.
[71] Skene, P. J., and Henikoff, S. (2017) An efficient targeted nuclease strategy for high-resolution mapping of DNA binding sites, Elife 6.

All patents, patent applications, and other publications, including GenBank Accession Numbers or equivalents, cited in this application are incorporated by reference in the entirety for all purposes.

INFORMAL SEQUENCE LISTING
KSHV LANA peptide amino acid sequence
SEQ ID NO: 1
WKFAVIFWGNDPYGLK
VGN73 peptide amino acid sequence
SEQ ID NO: 2
WKHAVIFWGNDPYGLKK
VGN73 peptide amino acid sequence (variant1)
SEQ ID NO: 3
WKHAVIFWGNDPYGLK
VGN73 peptide amino acid sequence (variant2)
SEQ ID NO: 4
WKHAVIFWGNDPYGL
VGN73 peptide amino acid sequence (variant3)
SEQ ID NO: 5
WKHAVIFWGNDPYG
VGN73 peptide amino acid sequence (variant4)
SEQ ID NO: 6
WKHAVIFWGNDPY
VGN73 peptide amino acid sequence (variant5)
SEQ ID NO: 7
WKHAVIFWGNDP
VGN73 peptide amino acid sequence (variant6)
SEQ ID NO: 8
WKHAVIFWGND
TAT peptide amino acid sequence
SEQ ID NO: 9
GRKKRRQRRRPQ
modified TAT amino acid sequence
SEQ ID NO: 10
{d-Arg}KKRR{ORN]RRR{β-Ala}
TAT-KSHV LANA fusion peptide amino acid sequence
SEQ ID NO: 11
{d-Arg}KKRR{ORN}RRR{β-Ala}WKFAVIFWGNDPYGLK
TAT-VGN73 fusion peptide amino acid sequence
SEQ ID NO: 12
(d-Arg}KKRR{ORN}RRR{β-Ala}WKHAVIFWGNDPYGLKK
TAT-VGN73 variant1 fusion peptide amino acid
sequence
SEQ ID NO: 13
{d-Arg}KKRR{ORN}RRR{β-Ala}WKHAVIFWGNDPYGLK
TAT-VGN73 variant2 fusion peptide amino acid
sequence
SEQ ID NO: 14
{d-Arg}KKRR{ORN}RRR{β-Ala}WKHAVIFWGNDPYGL
TAT-VGN73 variant3 fusion peptide amino acid
sequence
SEQ ID NO: 15
{d-Arg}KKRR{ORN}RRR{β-Ala}WKHAVIFWGNDPYG
TAT-VGN73 variant4 fusion peptide amino acid
sequence
SEQ ID NO: 16
{d-Arg}KKRR{ORN}RRR{β-Ala}WKHAVIFWGNDPY
TAT-VGN73 variant5 fusion peptide amino acid
sequence
SEQ ID NO: 17
{d-Arg}KKRR{ORN}RRR{β-Ala}WKHAVIFWGNDP
TAT-VGN73 variant6 fusion peptide amino acid
sequence
SEQ ID NO: 18
{d-Arg}KKRR{ORN}RRR{β-Ala}WKHAVIFWGND
VGN73 mutant peptide amino acid sequence
SEQ ID NO: 19
AKHAVISAGNDPYGLKK
TAT-VGN73 mutant fusion peptide amino acid
sequence
SEQ ID NO: 20
{d-Arg}KKRR{ORN}RRR{β-Ala}AKHAVISAGNDPYGLKK

1. A polypeptide comprising a CHD4-binding peptide, wherein the CHD4-binding peptide comprises the amino acid sequence set forth in SEQ ID NO:8, is no more than about 100 amino acids in length, and inhibits proliferation of a CHD4+ cell.

2. The polypeptide of claim 1, wherein the CHD4-binding peptide consists of the amino acid sequence set forth in any one of SEQ ID NOs:1-8.

3. The polypeptide of claim 1, consisting of SEQ ID NO:2 and one or more heterologous amino acid sequences.

4. The polypeptide of claim 1, preferably at the C-terminus and/or N-terminus of the polypeptide.

5. (canceled)

6. The polypeptide of claim 1, wherein the heterologous amino acid sequence is a TAT sequence; and wherein the TAT peptide comprises the amino acid sequence set forth in SEQ ID NO:9 or 10.

7. (canceled)

8. The polypeptide of claim 6, having the amino acid sequence set forth in any one of SEQ ID NOs:11-18.

9. The polypeptide of claim 1, further comprising a heterologous amino acid sequence wherein the heterologous amino acid sequence is an antibody or an antigen-binding fragment thereof.

10. The polypeptide of claim 9, wherein the antibody or fragment specifically binds a cell surface antigen on a CHD4+ cell.

11. The polypeptide of claim 9, wherein the CHD4-binding peptide and the heterologous amino acid sequence are connected by a peptide linker comprising one or more protease cleavage sites.

12-15. (canceled)

16. A polynucleotide encoding the polypeptide of claim 1.

17. (canceled)

18. A vector comprising the polynucleotide of claim 16.

19. A host cell comprising the polynucleotide of claim 16.

20. A pharmaceutical composition comprising (1) the polypeptide of claim 1.

21. (canceled)

22. A method of inhibiting CDH4+ cell proliferation, the method comprising contacting a CDH4+ cell with an effective amount of the polypeptide of claim 1.

23. The method of claim 22, wherein the CHD4+ cell is a cancer cell.

24. A method of treating a CDH4+ cancer in a subject, the method comprising administering to the subject an effective amount of the polypeptide of any one of claim 1.

25. The method of claim 24, wherein the cancer is leukemia or lymphoma.

26. The method of claim 25, wherein the cancer is acute myeloid leukemia (AML), primary effusion lymphoma (PEL), or multiple myeloma.

27. (canceled)

28. The method of claim 24, further comprising administering a chemotherapeutic agent to the subject.

29. The method of claim 28, wherein the chemotherapeutic agent is Sunitinib.
